(19) United States
(12) Patent Application Publication
(10) Pub. No.: US 2007/0155736 A1
(43) Pub. Date: Jul. 5, 2007

(54) FLUORINATED PHNO AND ANALOGUES THEREOF

(75) Inventor: Philip Seeman, Toronto (CA)

Correspondence Address: BERESKIN AND PARR, 40 KING STREET WEST, BOX 401, TORONTO, ON, M5H 3Y2, CA

(73) Assignee: Clara Inc., Toronto (CA)

(21) Appl. No.: 11/558,675

(22) Filed: Nov. 10, 2006

Related U.S. Application Data
(63) Continuation-in-part of application No. PCT/CA06/00191, filed on Feb. 10, 2005.

(51) Int. Cl. A61K 31/538; C07D 265/34

(52) U.S. Cl. 514/229.8; 544/101

(57) ABSTRACT

This application describes fluorinated (+)PHNO and analogs thereof, including radiolabeled analogs, compositions comprising these compounds and methods of using these compounds, for example, for identifying dopamine supersensitivity.

### FIELD OF THE INVENTION

The present invention relates to fluorinated PHNO and analogs thereof, and their corresponding radiolabelled compounds and their use in vitro and in vivo for binding to dopamine receptors for the treatment of dopamine-related disorders as well as for detecting functional D2 and/or D3 receptors and determining supersensitivity of the dopamine neurotransmission system in the human brain in health and disease, including psychosis, schizophrenia, Attention-deficit Hyperactivity Disorder (ADHD), addiction and Parkinson's disease.

### BACKGROUND OF THE INVENTION

Psychotic symptoms occur in many diseases, including schizophrenia and prolonged drug abuse. Although many chromosome regions and genes have been found associated with schizophrenia, no single gene of major effect has yet been identified. Nevertheless, regardless of the causes of psychosis, antipsychotic drugs are mostly effective in alleviating the symptoms. Because the clinical antipsychotic potencies of these drugs are directly related to their affinities for the dopamine D2 receptor (P. Seeman, et al. Proc. Nat. Acad. Sci, U.S.A. 72: 4376, 1975; P. Seeman, Can. J. Psychiatry 47: 27, 2002), this suggests that the properties of this receptor are disturbed in psychosis. It is uncertain whether the total density of D2 receptors in schizophrenia is elevated (A. L. Nordstrom, et al., Psychiatry Res. 61: 67,1995; A. Abi-Dargham et al. Proc. Nat. Acad. Sci, U.S.A. 72: 7673, 2000).

The more relevant question, however, is whether the functional state of D2, or the state of high-affinity for dopamine, D₂^{High} (S. R. George et al., Endocrinology 117: 690, 1985), is elevated, and this has not been investigated in schizophrenia or in any of the psychoses. There are two classes of dopamine receptors in the brain, type D1 and type D2. The dopamine receptors are part of the general family of G protein-linked receptors. A receptor which is linked to a G protein (of which there are many types) can exist in two states. These two states are referred to as the high affinity state and low affinity state. For example, in the case of the dopamine receptor, dopamine has a dissociation constant of 1.5 nM for the high-affinity state, or D2^{High}, and approximately 200-2000 nM for the low-affinity state, or D₂^{Low}. Depending on local conditions in vitro or in vivo, the two states can quickly convert into each other. Because the high-affinity state is considered the functional state (S. R. George et al., Endocrinology 117: 690, 1985), the process of “desensitization” occurs whenever the high-affinity state converts into the low-affinity state.

An increased number or elevated density of D2^{High} receptors would explain why individuals with schizophrenia or psychosis are supersensitive to dopamine-mimetics (J. A. Lieberman, et al. Psychopharmacology 91: 415, 1987).

Experimentally, dopamine-mimetic supersensitivity occurs after a neonatal lesion of the brain (S. K. Bhardwaj, et al., Neuroscience 122: 669, 2003); prolonged use of antipsychotics (T. F. Seeger, et al., Psychopharmacology 76: 182, 1982), ethanol or amphetamine (T. E. Robinson, K. C. Berridge, Addiction 95(Suppl. 2): S91, 2000); in gene knockouts of Dbh (dopamine beta-hydroxylase) (D. Weinshenker, et al., Proc. Nat. Acad. Sci., USA 99: 13873, 2002), dopamine D4 receptors (M. Rubinstein et al., Cell, 90: 1991, 1997), GRK6 (G protein-coupled receptor kinase 6 (R. R. Gainetdinov et al., Neuron 38: 291, 2003), or COMT (catechol-O-methyltransferase) (M. Huotari, et al., Psychopharmacology 172: 1, 2004); and in rats born by C-section (P. Boksa, et al., Exper. Neurol. 175: 388, 2002). While antipsychotics are known to elevate the density of dopamine D2 receptors by ˜25% above control levels, no such elevations occur in ethanol withdrawal (P. Seeman, et al., Synapse 52: 77, 2004), in amphetamine-sensitized animals (P. Seeman, et al. Synapse 46: 235, 2002), in GRK6 or COMT knockouts, or in rats born by C-section.

It has recently been found that, despite the absence of any elevation in total dopamine D2 receptors in the striata of amphetamine-sensitized animals, there is a dramatic 360% increase (P. Seeman, et al., Synapse 46, 235, 2002) in the density of D2^{High} states. It has more recently been found that the density or proportion of D2^{High} states is also invariably elevated in other conditions showing dopamine supersensitivity. This was found to be the case in studying the brain striata from many types of animals that are known to be dopamine supersensitive after treatment with either antipsychotics, quinpirole, ethanol or amphetamine, after a hippocampal lesion, or following four types of gene knockouts (P. Seeman, et al., Proc. Nat. Acad. Sci, USA 102:3513-1518, 2005).

The state of dopamine supersensitivity usually develops in early stages of dopamine-related diseases. In order to assess, treat, and follow the progress of such dopamine-related illnesses, there is a need to measure the state of dopamine supersensitivity in its various stages of progression.

Although there are many molecular mechanisms underlying dopamine supersensitivity, the common component is an increase in the number or proportion of D2 receptors in the high-affinity state for dopamine. The D2 receptor is the primary target for all antipsychotic drugs. There are five types of dopamine receptors in humans (D1, D2, D3, D4, and D5; P. Seeman, Neuropsychopharmacology 7: 261-284, 1992). Of these, only the D2 receptor is blocked by antipsychotic drugs in direct relation to their clinical antipsychotic potencies P. Seeman, T. Lee, M. Chau-Wong, K. Wong, Nature 261: 717-719, 1976). As stated by Su et al. (Arch. Gen. Psychiat. 54: 972-973, 1997), “ . . . no drug has yet been identified with antipsychotic action without a significant affinity for the dopamine D2 receptor.” Furthermore, the concentrations of antipsychotic drugs that block dopamine D2 receptors in vitro are identical to the concentrations of antipsychotic drugs that are found in the spinal fluid or in the plasma water (i.e., corrected for drug binding to the plasma proteins) of patients being successfully maintained on these drugs (P. Seeman, Canad. J. Psychiat. 47: 27-38, 2002). Therefore, because D2 is the common target for antipsychotic drugs, and because the high-affinity states of D2 receptors are elevated in dopamine supersensitivity, antipsychotic drugs can block, overcome or mask dopamine supersensitivity.

Normally, the proportion of D2 receptors which are in the high-affinity state, as measured in homogenized rat striatal tissue in vitro, is ˜10-20%, as monitored by the competition between dopamine and [³ H]spiperone. Several previous studies have examined whether the proportion of high-affinity states for D2 were elevated after antipsychotics or in GRK6 knockouts, with little if any significant change. This is because most laboratories have been using a dopamine/[³H]spiperone competition method. As shown elsewhere, however, dopamine (with its Kd of 1.75 nM) is not effective in competing versus the much more tightly bound [³H]spiperone (with its Kd of 60 pM), especially in 120 mM NaCl.

Using three methods (P. Seeman, et al., Proc. Natl. Acad. Sci. USA, 102:3513-3518, 2005), especially using dopamine/[³H]domperidone competition experiments, all types of animals that are supersensitive to dopamine, dopamine-mimetics or amphetamine were found to have a higher proportion of D2^{High} receptors. In fact, it was found that there was a 100% to 900% elevation in the proportion of D2 receptors in the high-affinity state in rats after neonatal hippocampal lesions, in rats after long-term treatment with antipsychotics, ethanol, or amphetamine, and in mice with gene knockouts of Dbh (dopamine beta-hydroxylase), D4 receptors, GRK6 (G protein-coupled receptor kinase 6) or COMT (catechol-O-methyltransferase), and in rats born by Caesarian-section.

In principle, the high-affinity state of the D2 receptor can be labeled by low concentrations of various radioactive dopamine agonists. For example, quinpirole is a dopamine agonist which is often used as a selective agonist for D2 receptors. Although quinpirole has a 250-fold selectivity for dopamine D2 receptors over D1 receptors (P. Seeman and J. M. Schaus, Eur. J. Pharmacol. 203: 105-109, 1991), its high dissociation constant of 5 nM makes it vulnerable to inhibition by endogenous dopamine. Moreover, radioactive quinpirole has high nonspecific binding, indicating that this compound binds to many other unidentified sites. As another example, (−)-N-[¹¹C]propyl-norapomorphine has been used to label D2 receptors (R. Narendran et al., Synapse 52: 188-208, 2004), however, it is known that propyl-norapomorphine has an equal affinity for the D1 and D2 receptors, with a 0.7 nM dissociation constant at both receptors.

A highly selective agonist for the high-affinity state of the D2 receptor is (+)-(4aR,10bR)-3,4,4a,5,6,10b-hexahydro-4-propyl-2H-naphth[1,2-b][1,4]oxazin-9-ol HCl, or (+)PHNO (also known as (+)-4-propyl-9-hydroxynaphthoxazine, MK458, L-647,339 or naxagolide). Although (+)PHNO is effective in alleviating Parkinson's disease (A. Lieberman et al., Clin. Neuropharmacol. 11: 191-200, 1988), its long-term use may lead to desensitization and a loss of clinical efficacy (J. M. Cedarbaum et al., Movement Disorders 5: 298-303, 1990).

The short-term use of tracer doses of radioactive (+)PHNO in radioimaging procedures is described in Applicant's co-pending PCT Patent Application Publication No. WO 2006/047861, filed concurrently with the present application.

There remains a need for further compounds which can be used to image dopamine D2 receptors, in particular D2High receptors, as well as a need to create more effective anti-Parkinson drugs which do not lose their efficacy with time.

### SUMMARY OF THE INVENTION

Described herein are compounds of Formula I, and pharmaceutically acceptable salts and solvates thereof: 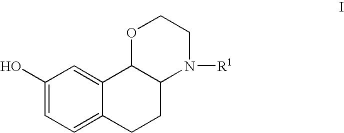
wherein R¹ is C₁₋₆alkyl in which one hydrogen atom on the alkyl chain is replaced with fluoro or radioactive fluoro.

The compounds of Formula I, including radioactive versions thereof, have good affinity and selectivity for the dopamine D2 receptors.

In addition to the high selectivity of the compounds of Formula I for D2 receptors, the dissociation constant of compounds of Formula I for D2 is lower than that of quinpirole, meaning that compounds of Formula I will bind more tightly to D2 and are less sensitive to endogenous dopamine which tends to interfere with the binding of any ligand to D2. The labelling of D2-like receptor by a compound of Formula I will also include D3 receptors to some extent. That is, the labelling of D₂High receptors would generally be admixed with the labelling of D3High receptors.

Accordingly the present invention also includes a method of labeling dopamine D2/3 receptors in vivo comprising administering an effective amount of a compound of the invention wherein R¹ is C₁₋₆alkyl in which one hydrogen atom on the alkyl chain is replaced with radioactive fluoro, to a subject in need thereof, and imaging an amount of said compound of the invention wherein R¹ is C₁₋₆alkyl in which one hydrogen atom on the alkyl chain is replaced with radioactive fluoro, in said subject. In an embodiment of the invention, the amount of the compound of the invention is imaged in the brain of said subject.

The present invention also includes a method for identifying and quantifying the extent of dopamine supersensitivity in the brain in various stages of a dopamine-related disease. In one of its embodiments, the method comprises injecting a trace amount of a compound of the invention wherein R¹ is C₁₋₆alkyl in which one hydrogen atom on the alkyl chain is replaced with radioactive fluoro intravenously into a subject, for example a human, and imaging, for example by means of positron emission tomography, the amount of the compound localized to the brain, in particular the caudate nucleus. This amount of the compound of the invention wherein R¹ is C₁₋₆alkyl in which one hydrogen atom on the alkyl chain is replaced with radioactive fluoro is correlated to the extent of dopamine sensitivity, with greater amounts indicating a greater extent of dopamine supersensitivity. In a further embodiment of the invention, twenty-four to forty-eight hours later, a second injection of a compound of the invention wherein R¹ is C₁₋₆alkyl in which one hydrogen atom on the alkyl chain is replaced with radioactive fluoro is given intravenously, contemporaneously with a low dose of a non-radiolabeled dopamine agonist or dopamine mimetic having an affinity or dissociation constant for the dopamine D_{2/3}^{High} receptor that is similar to a compound of the invention wherein R¹ is C₁₋₆alkyl in which one hydrogen atom on the alkyl chain is replaced with radioactive fluoro, for example between about 0.4 to about 3.0 nM for the high-affinity state of dopamine D2/3 receptors, and with a permeability across biological membranes that is similar to that for a compound of the invention wherein R¹ is C₁₋₆alkyl in which one hydrogen atom on the alkyl chain is replaced with radioactive fluoro. The contemporaneously administered dose of dopamine agonist or mimetic should be on the order of about 10 to about 50 times the dose of total active drug or active ingredient (i.e. compound of the invention) in the radiolabeled dose (radiolabeled and non-radiolabeled molecules), thus defining a baseline to determine the number of high-affinity states of D2/3 receptors in the same brain region. The difference between the brain image of the compound of the invention wherein R¹ is C₁₋₆alkyl in which one hydrogen atom on the alkyl chain is replaced with radioactive fluoro obtained without contemporaneous injection of non-radiolabeled dopamine agonist or mimetic and the corresponding image obtained with contemporaneous injection of non-radioactive dopamine agonist or mimetic is designated as the “specific binding” of the radioactive compounds of the invention. The amount of specific binding of a compound of the invention wherein R¹ is C₁₋₆alkyl in which one hydrogen atom on the alkyl chain is replaced with radioactive fluoro localized in a particular region of the brain is related to the extent of dopamine sensitivity of the brain, with higher than normal specific binding reflecting the presence of more high-affinity states of D2/3 receptors, with an associated significant dopamine supersensitivity in behaviour and supersensitivity to dopamine agonists.

Accordingly, the present invention relates to a method for determining an amount of dopamine D_{2/3}^{High} receptors comprising administering an effective amount of a compound of the invention wherein R¹ is C₁₋₆alkyl in which one hydrogen atom on the alkyl chain is replaced with radioactive fluoro to a subject and determining, by radioimaging, an amount of a compound of the invention wherein R¹ is C₁₋₆alkyl in which one hydrogen atom on the alkyl chain is replaced with radioactive fluoro in the subject's brain, or determining specific binding of the compound of the invention wherein R¹ is C₁₋₆alkyl in which one hydrogen atom on the alkyl chain is replaced with radioactive fluoro, in the subject's brain. The presence of a compound of the invention wherein R¹ is C₁₋₆alkyl in which one hydrogen atom on the alkyl chain is replaced with radioactive fluoro in the subject's brain indicates the presence of dopamine D2/3^{High} receptors, such the greater the amount of the compound, the greater the amount of dopamine D2/3^{High} receptors in that area. Further the specific binding of the compound of the invention wherein R¹ is C₁₋₆alkyl in which one hydrogen atom on the alkyl chain is replaced with radioactive fluoro in the subject's brain is correlated with the amount of dopamine D2/3^{High} receptors in that area, such that the greater the specific binding of the compound of the invention wherein R¹ is C₁₋₆alkyl in which one hydrogen atom on the alkyl chain is replaced with radioactive fluoro, the greater the number of D2/3^{High} receptors. In an embodiment of the present invention, the radiolabeled amount, or the specific binding, of the compound of the invention wherein R¹ is C₁₋₆alkyl in which one hydrogen atom on the alkyl chain is replaced with radioactive fluoro in the brain of the subject is compared to a control and if the amount or the specific binding is greater in the subject compared to the control then the subject is in a state of dopamine supersensitivity.

In a further embodiment of the present invention, there is included a method of determining an extent of dopamine supersensitivity in a subject comprising administering an effective amount of a compound of the invention wherein R¹ is C₁₋₆alkyl in which one hydrogen atom on the alkyl chain is replaced with radioactive fluoro to a subject and determining, by radioimaging, an amount of a compound of the invention wherein R¹ is C₁₋₆alkyl in which one hydrogen atom on the alkyl chain is replaced with radioactive fluoro in the subjects brain, or determining the specific binding of a compound of the invention wherein R¹ is C₁₋₆alkyl in which one hydrogen atom on the alkyl chain is replaced with radioactive fluoro in the subject's brain, wherein the greater the amount or the specific binding of the compound, the greater the number of D2/3^{High} receptors and wherein the greater the number of D2/3^{High} receptors, the greater the extent of dopamine supersensitivity.

The extent of dopamine supersensitivity is an important factor in the assessment of health and disease in a subject, for example, to assess, treat and/or follow the progress of any dopamine-related disorder.

The present invention also includes the use of a compound of the invention wherein R¹ is C₁₋₆alkyl in which one hydrogen atom on the alkyl chain is replaced with radioactive fluoro to determine an amount of dopamine D_{2/3}^{High} receptors in vivo as well as the use of radiolabeled to prepare a medicament to determine an amount of dopamine D2/3^{High} receptors in vivo.

Further, the present invention includes the use of a compound of the invention wherein R¹ is C₁₋₆alkyl in which one hydrogen atom on the alkyl chain is replaced with radioactive fluoro to determine an extent of dopamine supersensitivity in vivo as well as the use of a compound of the invention wherein R¹ is radioactive fluoro to prepare a medicament to determine an extent of dopamine supersensitivity in vivo.

The present invention also includes a method for conducting positron emission tomography (PET) of a subject comprising administering to the subject an effective amount of a compound of Formula I wherein R¹ is C₁₋₆alkyl in which one hydrogen atom on the alkyl chain is replaced with radioactive fluoro and measuring the distribution within the subject of the compound by PET.

The present invention further includes the use of a compound of the invention in in vitro assays for screening for compounds that have an affinity for the dopamine D2 or D3 receptors. In such assays, the compound of Formula I may be non-radiolabeled or radiolabeled.

Accordingly, the present invention further includes a method of screening for compounds that bind to the D2^{High} receptor or D3^{High} receptor comprising (a) combining a sample comprising the D2^{High} receptor or D₃^{High} receptor with a compound of the invention wherein R¹ is C₁₋₆alkyl in which one hydrogen atom on the alkyl chain is replaced with radioactive fluoro and test compound under conditions sufficient for binding of the compound and the test compound to the D2^{High} receptor or D3^{High} receptor; and (b) determining an amount of binding of the compound that is inhibited in the presence of the test compound. The invention also includes the use of compound of the invention wherein R¹ is C₁₋₆alkyl in which one hydrogen atom on the alkyl chain is replaced with radioactive fluoro to screen for compounds that bind to the D2^{High} receptors or D3^{High} receptor.

Fluorine-18 (¹⁸F) is the most attractive PET radionuclide (97% abundant) for radiolabeling because its 110 minute half-life allows sufficient time (3×110 minutes) for incorporation into the radiopharmaceutical and for purification of the final product suitable for human administration. Further, ¹⁸F can be prepared in curie quantities as fluoride ion for incorporation into the radiopharmaceutical in high (theoretical 1.7 Ci/nmol) specific activity by no-carrier added nucleophilic substitution reactions. Fluorine-18 is also the lowest energy positron emitter (0.635 MeV, 2.4 mm positron range) which afford the highest resolution images. Finally the 110 minute half-life allows sufficient time for regional distribution up to a 200 mile radius from the manufacturing site.

In another of its aspects, the present invention also includes a method of treating a dopamine-related disorder comprising administering to a subject in need thereof, an effective amount of a compound selected from a compound of Formula I′: 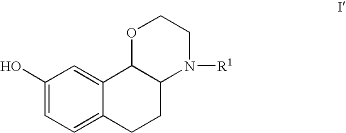
and pharmaceutically acceptable salts and solvates thereof, wherein R¹ is C₁₋₆alkyl in which one hydrogen atom on the alkyl chain is replaced with fluoro.

Also included is a use of a compound selected from a compound of Formula I′ wherein R¹ is C₁₋₆alkyl in which one hydrogen atom on the alkyl chain is replaced with fluoro, and pharmaceutically acceptable salts and solvates thereof, to treat a dopamine-related disorder and a use of a compound selected from a compound of Formula I′ wherein R¹ is C₁₋₆alkyl in which one hydrogen atom on the alkyl chain is replaced with fluoro, and pharmaceutically acceptable salts and solvates thereof, to prepare a medicament to treat a dopamine related disorder. In embodiments of the invention, the dopamine-related disorder is selected from Parkinson's disease, psychoses, schizophrenia, addiction, attention-deficit hyperactivity disorder (ADHD or ADD), adult attention-deficit disorder (AADD), depression, Huntington's disease and Progressive Supranuclear Palsy, in particular, Parkinson's disease.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present invention includes a compound selected from a compound of Formula I: 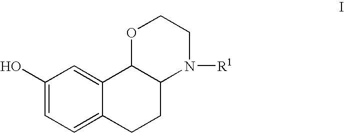
and pharmaceutically acceptable salts and solvates thereof,
wherein R¹ is C₁₋₆alkyl in which one hydrogen atom on the alkyl chain is replaced with fluoro or radioactive fluoro.

The term “alkyl” and “alkylene” as used throughout the present application includes straight and branched chain alkyl and alkylene groups, respectively.

The compounds of the invention include those in which R¹ is C₁₋₆alkyl in which one hydrogen atom on the alkyl chain is replaced with fluoro or radioactive fluoro. Accordingly, in an embodiment of the invention R¹ is selected from the group consisting of methyl, ethyl, n-propyl, isopropyl, n-butyl, s-butyl, isobutyl, t-butyl, n-pentyl, isopentyl, neopentyl, t-pentyl, 3-methylbutan-2-yl, 2-methylbutan-1-yl, n-hexyl, hexan-2-yl, 2-methylpentan-2-yl, 2,3-dimethylbutan-2-yl, 3,3-dimethylbutan-2-yl, 3-methylpentan-2-yl, 4-methylpentan-2-yl, 2,3-dimethylbutan-1-yl, 3,3-dimethylbutan-1-yl, hexan-2-yl, 2-methylpentan-1-yl, 3-methylpentan-1-yl, 4-methylpentan-1-yl and hexan-3-yl, wherein one hydrogen atom on R¹ is replaced with fluoro or radioactive fluoro. In a further embodiment of the invention, R¹ is selected from the group consisting of methyl, ethyl, n-propyl, isopropyl, n-butyl, s-butyl, isobutyl and t-butyl, wherein one hydrogen atom on R¹ is replaced with fluoro or radioactive fluoro. In a still further embodiment of the invention, R¹ is selected from the group consisting of methyl, ethyl, n-propyl, n-butyl, n-pentyl and n-hexyl, wherein one hydrogen atom on R¹ is replaced with fluoro or radioactive fluoro. In another further embodiment of the invention, R¹ is selected from the group consisting of ethyl, n-propyl and n-butyl, wherein one hydrogen atom on R¹ is replaced with fluoro or radioactive fluoro. In yet another further embodiment of the invention, R¹ is selected from the group consisting of ethyl and n-propyl, wherein one hydrogen atom on R¹ is replaced with fluoro or radioactive fluoro. In another embodiment of the invention R¹ is n-propyl wherein one hydrogen atom on R¹ is replaced with fluoro or radioactive fluoro.

In an embodiment of the invention, the hydrogen on R¹ that is replaced with fluoro or radioactive fluoro is a hydrogen atom on a terminal carbon atom. The term “terminal carbon atom” as used herein refers to the carbon atom in R¹ that is furthest away from the nitrogen atom to which R¹ is attached.

It is an embodiment of the invention that the hydrogen atom on R¹ is replaced with radioactive fluoro. It is a further embodiment of the invention that the radioactive fluoro is [¹⁸F].

It is another embodiment of the invention that the hydrogen atom on R¹ is replaced with fluoro (i.e. non-radioactive fluoro).

In an embodiment of the invention the compound of formula I is a compound selected from a compound of Formula Ia: 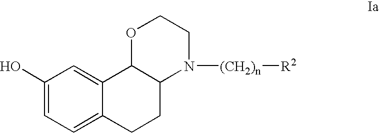
and pharmaceutically acceptable salts and solvates thereof,
wherein n is 1, 2, 3, 4, 5 or 6 and R² is fluoro or radioactive fluoro.

In an embodiment of the invention, n, in the compound of Formula Ia, is 1, 2, 3 or 4. In a further embodiment of the invention, n, in the compound of Formula Ia, is 2 or 3. In yet another embodiment, n, in the compound of Formula Ia, is 3.

In an embodiment of the invention, R², in the compound of Formula Ia, is radioactive fluoro, suitably [18F].

In an embodiment of the invention, R², in the compound of Formula Ia, is fluoro (i.e. non-radioactive fluoro).

In an embodiment of the invention, the compound of the invention is selected from the group consisting of:
[¹⁸F]-4-(3-fluoropropyl)-3,4,4a,5,6,10b-hexahydro-2H-naphtho[1,2-b][1,4]oxazin-9-ol;
[¹⁸F]-4-(2-fluoroethyl)-3,4,4a,5,6,10b-hexahydro-2H-naphtho[1,2-b][1,4]oxazin-9-ol;
[¹⁸F]-4-(4-fluorobutyl)-3,4,4a,5,6,10b-hexahydro-2H-naphtho[1,2-b][1,4]oxazin-9-ol;
[¹⁸F]-4-(5-fluoropentyl)-3,4,4a,5,6,10b-hexahydro-2H-naphtho[1,2-b][1,4]oxazin-9-o;
[¹⁸F]-4-(6-fluorohexyl)-3,4,4a,5,6,10b-hexahydro-2H-naphtho [1,2-b][1,4]oxazin-9-ol;
[¹⁸F]-(4aR,10bR)-4-(2-fluoroethyl)-3,4,4a,5,6,10b-hexahydro-2H-naphtho[1,2-b][1,4]oxazin-9-ol;
[¹⁸F]-(4aR,10bR)-4-(3-fluoropropyl)-3,4,4a,5,6,10b-hexahydro-2H-naphtho[1,2-b][1,4]oxazin-9-ol;
[¹⁸F]-(4aR,10bR)-4-(4-fluorobutyl)-3,4,4a,5,6,10b-hexahydro-2H-naphtho[1,2-b][1,4]oxazin-9-ol;
[¹⁸F]-(4aR,10bR)-4-(5-fluoropentyl)-3,4,4a,5,6,10b-hexahydro-2H-naphtho[1,2-b][1,4]oxazin-9-o;
[¹⁸F]-(4aR,10bR)-4-(6-fluorohexyl)-3,4,4a,5,6,10b-hexahydro-2H-naphtho[1,2-b][1,4]oxazin-9-ol;
4-(3-fluoropropyl)-3,4,4a,5,6,10b-hexahydro-2H-naphtho[1,2-b][1,4]oxazin-9-ol;
4-(2-fluoroethyl)-3,4,4a,5,6,10b-hexahydro-2H-naphtho[1,2-b][1,4]oxazin-9-ol;
4-(4-fluorobutyl)-3,4,4a,5,6,10b-hexahydro-2H-naphtho[1,2-b][1,4]oxazin-9-ol;
4-(5-fluoropentyl)-3,4,4a,5,6,10b-hexahydro-2H-naphtho [1,2-b][1,4]oxazin-9-ol;
4-(6-fluorohexyl)-3,4,4a,5,6,10b-hexahydro-2H-naphtho[1,2-b][1,4]oxazin-9-ol;
(4aR,10bR)-4-(2-fluoroethyl)-3,4,4a,5,6,10b-hexahydro-2H-naphtho[1,2-b][1,4]oxazin-9-ol;
(4aR,10bR)-4-(3-fluoropropyl)-3,4,4a,5,6,10b-hexahydro-2H-naphtho[1,2-b][1,4]oxazin-9-ol;
(4aR,10bR)-4-(4-fluorobutyl)-3,4,4a,5,6,10b-hexahydro-2H-naphtho[1,2-b][1,4]oxazin-9-ol;
(4aR,10bR)-4-(5-fluoropentyl)-3,4,4a,5,6,10b-hexahydro-2H-naphtho[1,2-b][1,4]oxazin-9-o; and
(4aR,10bR)-4-(6-fluorohexyl)-3,4,4a,5,6,10b-hexahydro-2H-naphtho[1,2-b][1,4]oxazin-9-ol and
pharmaceutically and radiopharmaceutically acceptable salts thereof.

In another embodiment of the invention, the compound of the invention is selected from the group consisting of:
[¹⁸F]-4-(3-fluoropropyl)-3,4,4a,5,6,10b-hexahydro-2H-naphtho[1,2-b][1,4]oxazin-9-ol;
[¹⁸F]-4-(2-fluoroethyl)-3,4,4a,5,6,10b-hexahydro-2H-naphtho[1,2-b][1,4]oxazin-9-ol;
[¹⁸F]-(4aR,10bR)-4-(2-fluoroethyl)-3,4,4a,5,6,10b-hexahydro-2H-naphtho[1,2-b][1,4]oxazin-9-ol; and
[¹⁸F]-(4aR,10bR)-4-(3-fluoropropyl)-3,4,4a,5,6,10b-hexahydro-2H-naphtho[1,2-b][1,4]oxazin-9-ol,
and radiopharmaceutically acceptable salts thereof.

In yet a further embodiment of the invention, the compound of the invention is selected from the group consisting of:
4-(3-fluoropropyl)-3,4,4a,5,6,10b-hexahydro-2H-naphtho[1,2-b][1,4]oxazin-9-ol;
4-(2-fluoroethyl)-3,4,4a,5,6,10b-hexahydro-2H-naphtho[1,2-b][1,4]oxazin-9-ol;
(4aR,10bR)-4-(2-fluoroethyl)-3,4,4a,5,6,10b-hexahydro-2H-naphtho[1,2-b][1,4]oxazin-9-ol; and
(4aR,10bR)-4-(3-fluoropropyl)-3,4,4a,5,6,10b-hexahydro-2H-naphtho[1,2-b][1,4]oxazin-9-ol,
and pharmaceutically acceptable salts thereof.

The novel compounds of the invention have at least two asymmetric carbon atoms, 4a and 1a, where the morpholino ring is fused to the tetrahydronaphthalene ring. The stereoisomer having a trans configuration between these two asymmetric carbon atoms has greater dopaminergic activity. Therefore the present invention includes compounds of Formula I as a substantially pure diastereomer having the trans configuration between the 1a and 4a carbon atoms. This configuration is referred to as the (+)-trans configuration. This invention also includes the individual diastereomers and enantiomers and mixtures thereof, in all proportions, including racemic mixtures containing about equal amounts of all stereoisomers. In an embodiment of the invention, the compounds of the invention include the (+)-trans diastereomer, although it is to be understood that such compounds of the invention may also contain certain amounts (e.g. less than 20%, suitably less than 10%, more suitably less than 5%) of compounds of the invention having alternate stereochemistry. For example, a compound of the invention having the (+)-trans configuration between carbons 1a and 4a, may contain less than 20%, suitably less than 10%, more suitably less than 5%, of a compound of the invention having the (−)-trans configuration.

The term “compound(s) of the invention” as used herein means compound(s) of Formula I and 1a, and/or pharmaceutically acceptable salts and/or solvates thereof.

It is to be clear that the present invention includes pharmaceutically acceptable salts and solvates of compounds of Formula I and Ia, and mixtures comprising two or more of the compounds of Formula I, compounds of Formula Ia, pharmaceutically acceptable salts of the compounds of Formula I, pharmaceutically acceptable salts of the compounds of Formula Ia, pharmaceutically acceptable solvates of compounds of Formula I and pharmaceutically acceptable solvates of compounds of Formula Ia.

The term “pharmaceutically acceptable” means compatible with the treatment of animals, in particular, humans.

The term “pharmaceutically acceptable salt” means an acid addition salt which is suitable for or compatible with the treatment of patients.

The term “pharmaceutically acceptable acid addition salt” as used herein means any non-toxic organic or inorganic salt of any base compound of the invention. Basic compounds of the invention that may form an acid addition salt include those having a basic nitrogen. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Such salts may exist in either a hydrated, solvated or substantially anhydrous form. In general, the acid addition salts of the compounds of the invention are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the invention, for laboratory use, or for subsequent conversion to a pharmaceutically acceptable acid addition salt. In an embodiment of the invention, the pharmaceutically acceptable salt is a hydrochloride salt.

The term “solvate” as used herein means a compound of the invention, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

In accordance with another aspect of the present invention, the compounds of the invention can be prepared by processes analogous to those established in the art. Therefore, compounds of the invention may be prepared, for example, by the reaction sequence shown in Scheme 1: 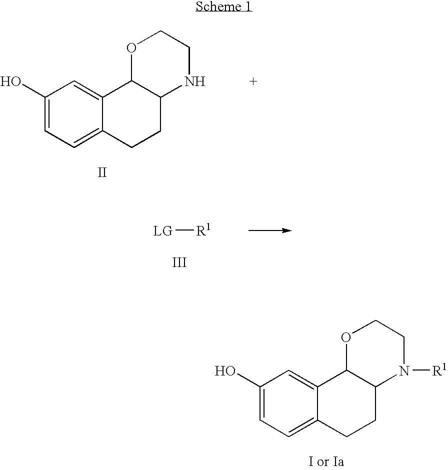
Therefore compounds of Formula II, may be reacted with compounds of Formula III, wherein R¹ is defined in Formula I and LG is a suitable leaving group such as halo, for example iodo or bromo, in the presence of a suitable base under standard nucleophilic substitution reaction conditions to provide compounds for Formula I or Ia.

Alternatively, compounds of Formula I or Ia wherein the carbon atom of R¹ that is attached to the nitrogen has two hydrogen atoms attached thereto, may be prepared as shown in Scheme 2. Accordingly compounds of Formula II may be reacted with compounds of Formula IV, wherein R³ is C₁₋₅alkyl in which one hydrogen atom on the alkyl chain is replaced with fluoro or radioactive fluoro and A is a suitable activating group, for example chloro, an acyloxy group or an activating group of suitable peptide coupling agent, to provide compounds of Formula V, wherein R³ is C₁₋₅alkyl in which one hydrogen atom on the alkyl chain is replaced with fluoro or radioactive fluoro, which may be reduced using a suitable reducing agent, for example a metal hydride such as lithium aluminum hydride, to provide compounds of Formula I or Ia wherein the carbon atom of R¹ that is attached to the nitrogen has two hydrogen atoms attached thereto and R¹ is C₁₋₆alkyl in which one hydrogen atom on the alkyl chain is replaced with fluoro or radioactive fluoro.

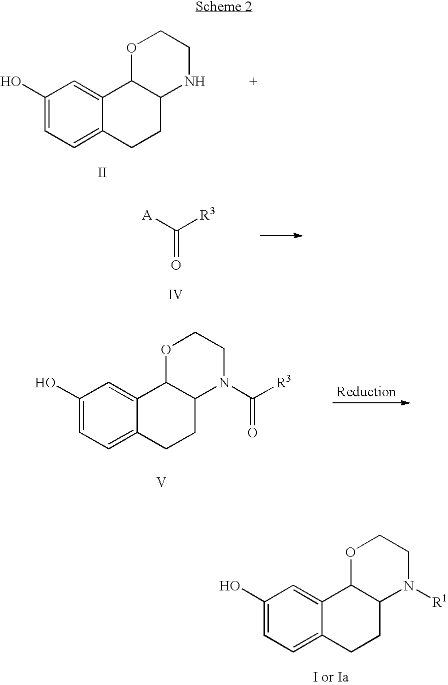

Compounds of Formula II, III and IV are either commercially available or may be prepared using methods known in the art. For example, the preparation of compounds of Formula II, including the individual stereoisomers, is described in EP 0080115A, U.S. Pat. No. 4,420,480 and U.S. Pat. No. 4,758,661.

In some cases, the methods outlined in Schemes 1 and 2 above may be modified to incorporate the use of protecting groups. For example, the hydroxyl group on the compound of Formula II may first be protected with a suitable protecting group which may be removed at the end of the synthesis to provide compounds of Formula I or Ia. Suitable protecting groups for the chemistries outlined above would be known to those skilled in the art, for example with reference to “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999. Suitable protecting groups are stable to basic conditions, for example alkyl ether protecting groups, such as methoxy methyl (MOM).

The incorporation of radioactive fluorine atoms into the compounds of Formula I or Ia may be performed using techniques known in the art, for example, using reagents of Formula III or IV in which R¹ is radioactive fluoro in the methods shown in Schemes 1 or 2. Such reagents may be prepared using methods known in the art (see, for example, Shiue, C.-Y. et al. J. Labeled Comp. Radiopharm. 24:56, 1987; Wilson, A. A. et al. Appl Radiat Isotopes, 46:765, 1995; Wilson, A. A. et al. Nucl Med Biol, 23:487, 1996). Reagents of Formula III or IV, wherein R¹ incorporates a radioactive fluoro are available by reaction of, for example, the corresponding tosylates (or other suitable leaving group) with a nucleophilic radioactive fluorinating reagent, such K[¹⁸F]/K222 or tetraalkyl ammonium salts incorporating radioactive fluoride (Culbert, P. A. et al. Appl. Radiat. Isotopes, 46:887, 1995; Mock, B. H. et al. Nucl. Med. Biol. 23:497, 1996). For reagents of Formula III, two leaving groups having different reactivity may be used, or alternatively, one leaving group may be replaced by a suitable protecting group that may be converted to a leaving group after incorporation of the radioactive fluorine.

Alternatively, the radioactive fluorine atom may be incorporated at the end of the synthesis, for example as shown in Scheme 3. Therefore a compound of Formula II may be reacted with one equivalent of a compound of Formula VI, wherein R⁴ is C₁₋₆alkylene and LG and X are suitable leaving groups, in the presence of a base under standard nucleophilic substitution reaction conditions to provide compounds of Formula VII, wherein R⁴ is C₁₋₆alkylene and X is a suitable leaving group. The compounds of Formula VII are then treated with a nucleophilic radioactive fluorinating reagent to provide, compounds of Formula I, or Ia wherein R¹ is C₁₋₆alkyl wherein one hydrogen atom on the alkyl chain is replaced by radioactive fluoro. For the reagents of Formula VI, two leaving groups having different reactivity may be used, or alternatively, leaving group X may be replaced by a suitable protecting group that may be converted to a leaving group after reaction with the compound of Formula II.

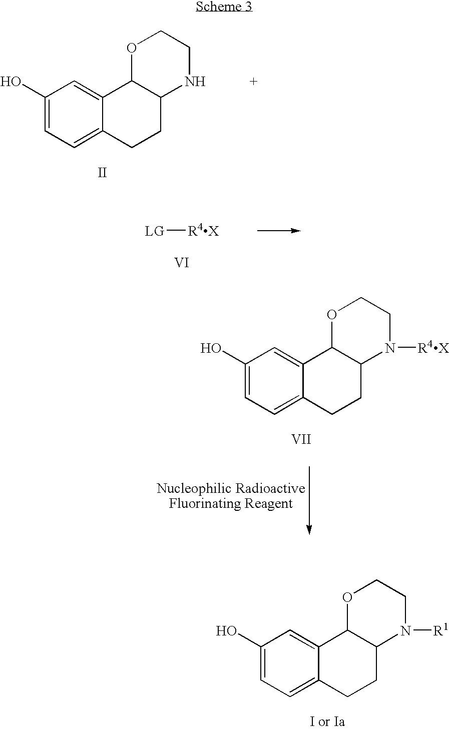

Alternatively, the radioactive fluorine atom may be incorporated at the end of the synthesis for compounds of Formula I or Ia wherein the carbon atom of R¹ that is attached to the nitrogen has two hydrogen atoms attached thereto as shown in Scheme 4. Accordingly, compounds of Formula II, may be reacted with compounds of Formula VIII, wherein R⁵ is C₁₋₅alkylene, X is a suitable leaving group and A is a suitable activating group, for example chloro, an acyloxy group or an activating group of suitable peptide coupling agent, to provide compounds of Formula IX, wherein R⁵ is C₁₋₅alkylene and X is a suitable leaving group, which may be reduced using a suitable reducing agent, for example a metal hydride such as lithium aluminum hydride, to provide compounds of Formula XI, wherein R⁵ is C₁₋₅alkylene and X is a suitable leaving group. The compounds of Formula XI are then treated with a nucleophilic radioactive fluorinating reagent to provide compounds of Formula I or Ia, wherein the carbon atom of R¹ that is attached to the nitrogen has two hydrogen atoms attached thereto and R¹ is C₁₋₆alkyl in which one hydrogen atom on the alkyl chain is replaced with radioactive fluoro.

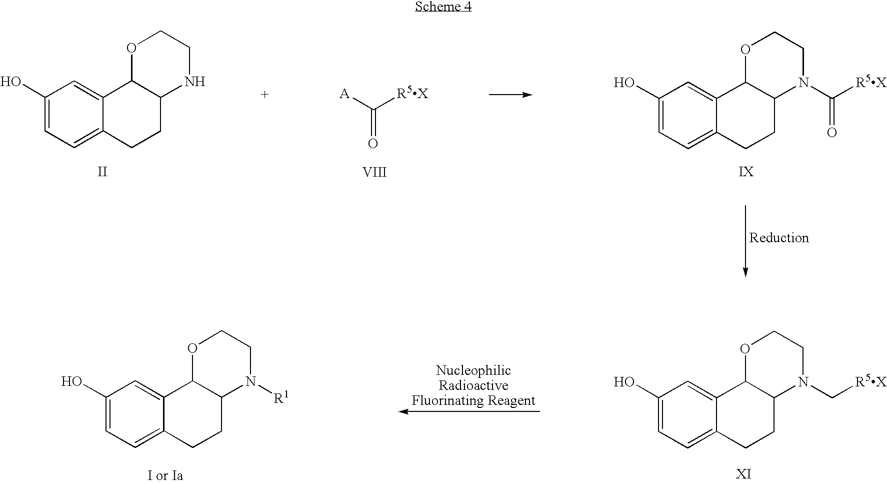

In some cases, the methods outlined in Schemes 3 and 4 above may be modified to incorporate the use of protecting groups. For example, the hydroxyl group on the compound of Formula II may first be protected with a suitable protecting group which may be removed at the end of the synthesis to provide compounds of Formula I or Ia. Suitable protecting groups for the chemistries outlined above would be known to those skilled in the art, for example with reference to “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999. Suitable protecting groups are stable to basic conditions, for example alkyl ether protecting groups, such as methoxy methyl (MOM).

The formation of a desired compound salt is achieved using standard techniques. For example, the neutral compound is treated with an acid in a suitable solvent and the formed salt is isolated by filtration, extraction, recrystallization or any other suitable method.

The formation of solvates of the compounds of the invention will vary depending on the compound and the solvate. In general, solvates are formed by dissolving the compound in the appropriate solvent and isolating the solvate by cooling or using an antisolvent. The solvate is typically dried or azeotroped under ambient conditions.

Compounds of the invention have good affinity for the dopamine D2 receptors. For example, the dissociation constant, K_{D}, at the D2^{High} receptors (using [³H]-domperidone) for compounds of Formula I wherein R¹ is —(CH₂)₂₋₆-F ranges from about 0.45 to about 50 nM. The compounds of the invention also have good affinity for the dopamine D3 receptors. For example, the dissociation constant, K_{D} at the rat cloned D3^{High} receptors (using [³H]-raclopride) for compounds of Formula I wherein R¹ is —(CH₂)₂₋₆-F ranges from about 0.4 to about 50 nM. It is expected that the compounds of Formula I will also have a selectivity for the dopamine D2/3 receptors that will permit their effective use to label D2/3 ^{High} receptors in vivo. It is herein submitted that the short-term use of tracer doses of a compound of the invention, wherein R¹ is C₁₋₆alkyl in which one hydrogen atom on the alkyl chain in replaced with radioactive fluoro, in radioimaging procedures will be of diagnostic and therapeutic importance in dopamine-related disorders.

Accordingly the present invention also includes a method of labeling dopamine D2/3 receptors in vivo comprising administering an effective amount of a compound of the invention wherein R¹ is C₁₋₆alkyl in which one hydrogen atom on the alkyl chain is replaced with radioactive fluoro, to a subject in need thereof, and imaging an amount of said compound of the invention wherein R¹ is C₁₋₆alkyl in which one hydrogen atom on the alkyl chain is replaced with radioactive fluoro, in said subject. In an embodiment of the invention, the amount of the compound of the invention is imaged in the brain of said subject.

The present invention also includes a method for identifying and quantifying the extent of dopamine supersensitivity in the brain in various stages of a dopamine-related disease. In one of its embodiments, the method comprises injecting a trace amount of a compound of the invention wherein R¹ is C₁₋₆alkyl in which one hydrogen atom on the alkyl chain is replaced with radioactive fluoro intravenously into a subject, for example a human, and imaging, for example by means of positron emission tomography, the amount of the compound localized to the brain, in particular the caudate nucleus. This amount of the compound of the invention wherein R¹ is C₁₋₆alkyl in which one hydrogen atom on the alkyl chain is replaced with radioactive fluoro is correlated to the extent of dopamine sensitivity, with greater amounts indicating a greater extent of dopamine supersensitivity. In a further embodiment of the invention, twenty-four to forty-eight hours later, a second injection of a compound of the invention wherein R¹ is C₁₋₆alkyl in which one hydrogen atom on the alkyl chain is replaced with radioactive fluoro is given intravenously, contemporaneously with a low dose of a non-radiolabeled dopamine agonist or dopamine mimetic having an affinity or dissociation constant for the dopamine D2/3^{High} receptor that is similar to a compound of the invention wherein R¹ is C₁₋₆alkyl in which one hydrogen atom on the alkyl chain is replaced with radioactive fluoro, for example between about 0.4 to about 3.0 nM for the high-affinity state of dopamine D2/3 receptors, and with a permeability across biological membranes that is similar to that for a compound of the invention wherein R¹ is C₁₋₆alkyl in which one hydrogen atom on the alkyl chain is replaced with radioactive fluoro. The contemporaneously administered dose of dopamine agonist or mimetic should be on the order of about 10 to about 50 times the dose of total active drug or active ingredient (i.e. compound of the invention) in the radiolabeled dose (radiolabeled and non-radiolabeled molecules), thus defining a baseline to determine the number of high-affinity states of D2/3 in the same brain region. The difference between the brain image of the compound of the invention wherein R¹ is C₁₋₆alkyl in which one hydrogen atom on the alkyl chain is replaced with radioactive fluoro obtained without contemporaneous injection of non-radiolabeled dopamine agonist or mimetic and the corresponding image obtained with contemporaneous injection of non-radioactive dopamine agonist or mimetic is designated as the “specific binding” of the radioactive compound of the invention. The amount of specific binding of a compound of the invention wherein R¹ is C₁₋₆alkyl in which one hydrogen atom on the alkyl chain is replaced with radioactive fluoro localized in a particular region of the brain is related to the extent of dopamine sensitivity of the brain, with higher than normal specific binding reflecting the presence of more high-affinity states of D2/3 receptors, with an associated significant dopamine supersensitivity in behaviour and supersensitivity to dopamine agonists.

Accordingly, the present invention relates to a method for determining an amount of dopamine D2/3 ^{High} receptors comprising administering an effective amount of a compound of the invention wherein R¹ is C₁₋₆alkyl in which one hydrogen atom on the alkyl chain is replaced with radioactive fluoro to a subject and determining, by radioimaging, an amount of a compound of the invention wherein R¹ is C₁₋₆alkyl in which one hydrogen atom on the alkyl chain is replaced with radioactive fluoro in the subject's brain, or determining specific binding of the compound of the invention wherein R¹ is C₁₋₆alkyl in which one hydrogen atom on the alkyl chain is replaced with radioactive fluoro, in the subject's brain. The presence of a compound of the invention wherein R¹ is C₁₋₆alkyl in which one hydrogen atom on the alkyl chain is replaced with radioactive fluoro in the subject's brain indicates the presence of dopamine D_{2/3}^{High} receptors, such the greater the amount of the compound, the greater the amount of dopamine D_{2/3}^{High} receptors in that area. Further the specific binding of the compound of the invention wherein R¹ is C₁₋₆alkyl in which one hydrogen atom on the alkyl chain is replaced with radioactive fluoro in the subject's brain is correlated with the amount of dopamine D2/3^{High} receptors in that area, such that the greater the specific binding of the compound of the invention wherein R¹ is C₁₋₆alkyl in which one hydrogen atom on the alkyl chain is replaced with radioactive fluoro, the greater the number of D2/3^{High} receptors. In an embodiment of the present invention, the radiolabeled amount, or the specific binding, of the compound of the invention wherein R¹ is C₁₋₆alkyl in which one hydrogen atom on the alkyl chain is replaced with radioactive fluoro in the brain of the subject is compared to a control and if the amount or the specific binding is greater in the subject compared to the control then the subject is in a state of dopamine supersensitivity.

The term “D2/3 receptors” and “D2/3^{High} receptors” as used herein means the “dopamine D2 receptor and/or dopamine D3 receptor” and “dopamine D2^{High} receptor and/or dopamine D3^{High} receptor”, respectively.

The term an “effective amount” as used herein is that amount sufficient to effect desired results, including clinical results, and, as such, an “effective amount” depends upon the context in which it is being applied. For example, in the context of administering an “effective amount” of a compound of the invention wherein R¹ is C₁₋₆alkyl in which one hydrogen atom on the alkyl chain is replaced with radioactive fluoro, an effective amount is, for example, an amount sufficient to generate an image to achieve labeling and/or quantification of the D2/3 receptors, including the D2/3^{high} receptors, in the brain of the subject.

The term “subject” as used herein includes all members of the animal kingdom including human. The subject is preferably a human.

The term “specific binding” as used herein is the difference in the amount or density of a compound of the invention wherein R¹ is C₁₋₆alkyl in which one hydrogen atom on the alkyl chain is replaced with radioactive fluoro, bound in a specific region of the subject's brain when the radioactive compound of the invention is administered alone and the amount of a compound of the invention wherein R¹ is C₁₋₆alkyl in which one hydrogen atom on the alkyl chain is replaced with radioactive fluoro bound in that same region when the radioactive compound of the invention is administered contemporaneously with a non-radiolabeled dopamine agonist or mimetic. The non-radiolabeled dopamine agonist should have an affinity or dissociation constant for the dopamine D2/3^{High} receptor that is similar to the compound of the invention wherein R¹ is C₁₋₆alkyl in which one hydrogen atom on the alkyl chain is replaced with radioactive fluoro, for example between about 0.4 to about 3.0 nM for the high-affinity state of dopamine D2/3 receptors, and a permeability across biological membranes that is similar to that for the compound of the invention wherein R¹ is C₁₋₆alkyl in which one hydrogen atom on the alkyl chain is replaced with radioactive fluoro. Accordingly, in an embodiment of the invention, the specific binding a compound of the invention wherein R¹ is C₁₋₆alkyl in which one hydrogen atom on the alkyl chain is replaced with radioactive fluoro is determined by:

(a) administering an effective amount of a compound of the invention wherein R¹ is C₁₋₆alkyl in which one hydrogen atom on the alkyl chain is replaced with radioactive fluoro to a subject and observing an amount or density of the compound in the subject's brain;

(b) allowing a suitable amount of time to pass for spontaneous decay of the compound of the invention administered in (a);

(c) contemporaneously administering an effective amount of the compound of the invention and an effective amount of a suitable non-radiolabeled dopamine agonist or dopamine-mimetic and observing an amount or density the compound of the invention in the subject's brain; and

(d) determining a difference between the amount or density of the compound of the invention in (a) and the amount or density of the compound of the invention in (c), wherein said difference is the specific binding of the compound of the invention.

A suitable time for the spontaneous decay of the compound of the invention wherein R¹ is C₁₋₆alkyl in which one hydrogen atom on the alkyl chain is replaced with radioactive fluoro in step (b) will depend on the identity of the radiolabel and its specific half life. A person skilled in the art would readily be able to determine this time.

In a further embodiment of the present invention, the amount or density of radiolabeled compound in steps (a) and (c) is determined in specific regions of the subject's brain. For example, the amount or density of the radiolabeled compound of the invention, may be observed in the striatum, the caudate nucleus, the putamen regions and/or the globus pallidus. The region used in (a) will be the same as that used in (c).

D2 (or D2^{High}) receptors are generally found in the putamen, the caudate nucleus, the nucleus accumbens, the anterior pituitary, the substantia nigra, the cerebral cortex and in the globus pallidus. D3 (or D3^{High}) is highly localized in the “ventral region of Tsai” (in the midbrain) and in the ventral region of the globus pallidus. More specifically, there is a mixed population of two-thirds D2 and one-third D3 dopamine receptors in the ventral putamen, the ventral caudate, and the globus pallidus (Seeman P, Wilson A, Gmeiner P, Kapur S.Dopamine D2 and D3 receptors in human putamen, caudate nucleus, and globus pallidus.Synapse. Sep. 1, 2000;60(3):205-21 1). Accordingly, a person skilled in the art, when utilizing a compound of the invention wherein R¹ is C₁₋₆alkyl in which one hydrogen atom on the alkyl chain is replaced with radioactive fluoro to observe the amount of dopamine receptors in a specific region of the brain, will be able to determine whether dopamine D2^{High}, dopamine D3^{High} or both dopamine D2^{High} and D3^{High} receptors are being observed.

The terms “determining” and “observing”, or synonyms thereof, are meant to include both qualitative and quantitative determinations of the amount or density of D2/3 ^{High} receptors localized brain or in the specified areas of the brain.

The term “dopamine mimetic” refers to any compound that acts like dopamine or causes a release of dopamine.

Suitable non-radiolabeled dopamine agonists or mimetics include, but are not limited to compounds of the invention wherein R¹ is C₁₋₆alkyl in which one hydrogen atom on the alkyl chain is replaced with fluoro, (+)-PHNO, amphetamine and apomorphine, and congeners thereof, for example, N-propyl-norapomorphine and various aminotetralins such as dihydroxy-2-dimethyl-aminotetralin. In an embodiment of the present invention, the non-radioactive dopamine agonist or mimetic is F-(+)PHNO, (+)PHNO, apomorphine or N-propyl-norapomorphine, in particular F-(+)PHNO, (+)PHNO or apomorphine. The dopamine mimetic will be selected based on whether dopamine D2^{High}, dopamine D3^{High} or both dopamine D2^{High} and D3^{High} receptors are being observed as would be known to a person skilled in the art. For example if only dopamine D2^{High} receptors are being observed, then the dopamine mimetic should have an affinity or dissociation constant for the dopamine D2^{High} receptor that is similar to that of the compound of the invention. Likewise, if only dopamine D3^{High} receptors are being observed, then the dopamine mimetic should have an affinity or dissociation constant for the dopamine D3^{High} receptor that is similar to that of the compound of the invention and if both dopamine D2^{High} and D3^{High} receptors are being observed, then the dopamine mimetic should have an affinity or dissociation constant for both the dopamine D2^{High} and D3^{High} receptors that is similar to those of the compound of the invention.

The contemporaneously administered dose or effective amount of dopamine agonist or mimetic should be on the order of about 10 to about 50 times the dose or effective amount of total active drug or active ingredient in the compound of the invention wherein R¹ is C₁₋₆alkyl in which one hydrogen atom on the alkyl chain is replaced with radioactive fluoro dose (radiolabeled and non-radiolabeled molecules), thus defining a baseline to determine the number of high-affinity states of D2/3 in the same brain region.

As used herein, “administered contemporaneously” means that the two substances are administered to a subject such that they are both biologically active in the subject at the same time. In particular embodiments, two substances will be administered substantially simultaneously, i.e. within minutes of each other, or in a single composition that comprises both substances.

The amount or specific binding of a compound of the invention wherein R¹ is C₁₋₆alkyl in which one hydrogen atom on the alkyl chain is replaced with radioactive fluoro in the subject's brain may be determined or observed, using any known technique to detect or image radioactive compounds in vivo. In an embodiment of the invention, the amount or the specific binding of a compound of the invention wherein R¹ is C₁₋₆alkyl in which one hydrogen atom on the alkyl chain is replaced with radioactive fluoro in the subject's brain is determined or observed using positron emission tomography (PET).

As mentioned above, the amount or the specific binding of a compound of the invention wherein R¹ is C₁₋₆alkyl in which one hydrogen atom on the alkyl chain is replaced with radioactive fluoro in the brain of the subject is compared to a control and if the amount or the specific binding is greater in the subject compared to the control then the subject is in a state of dopamine supersensitivity. As used herein, the term “control” means the amount or the specific binding of a compound of the invention wherein R¹ is C₁₋₆alkyl in which one hydrogen atom on the alkyl chain is replaced with radioactive fluoro that would be in the brain under standard or normal conditions. By “standard” or “normal” conditions it is meant in the absence of disease, injury, medication and/or substance (i.e. drug or alcohol) abuse, or any other factor that would affect the amount of D2^{High} receptors in the brain. For example, a control may be a subject that does not have any psychiatric illness of drug-induced illness. The term “greater” refers to any detectable increase in the specific binding of a compound of the invention wherein R¹ is C₁₋₆alkyl in which one hydrogen atom on the alkyl chain is replaced with radioactive fluoro in the brain of the subject compared to the control.

In a further embodiment of the present invention, there is included a method of determining an extent of dopamine supersensitivity in a subject comprising administering an effective amount of a compound of the invention wherein R¹ is C₁₋₆alkyl in which one hydrogen atom on the alkyl chain is replaced with radioactive fluoro to a subject and determining, by radioimaging, an amount of a compound of the invention wherein R¹ is C₁₋₆alkyl in which one hydrogen atom on the alkyl chain is replaced with radioactive fluoro in the subjects brain, or determining the specific binding of a compound of the invention wherein R¹ is C₁₋₆alkyl in which one hydrogen atom on the alkyl chain is replaced with radioactive fluoro in the subject's brain, wherein the greater the amount or the specific binding of the compound, the greater the number of D2/3^{High} receptors and wherein the greater the number of D2/3 ^{High} receptors, the greater the extent of dopamine supersensitivity.

Since, the presence of a compound of the invention wherein R¹ is C₁₋₆alkyl in which one hydrogen atom on the alkyl chain is replaced with radioactive fluoro in the subject's brain, indicates the presence of dopamine D2/3^{High} receptors and the amount or the specific binding of a compound of the invention wherein R¹ is C₁₋₆alkyl in which one hydrogen atom on the alkyl chain is replaced with radioactive fluoro in the various regions of the subject's brain is correlated with the amount of dopamine D2/3^{High} receptors in that area, such that the greater the amount or the specific binding of a compound of the invention wherein R¹ is C₁₋₆alkyl in which one hydrogen atom on the alkyl chain is replaced with radioactive fluoro, the greater the number of D2/3^{High} receptors, the method of the present invention can be used to determine if a subject is in a state of dopamine supersensitivity. The extent of dopamine supersensitivity is an important factor in the assessment of health and disease in a subject, for example, to assess, treat and/or follow the progress of any dopamine-related disorder. If a subject has elevated levels of dopamine D2/3^{High} receptors in their brain compared to a control, then they may be considered to have dopamine supersensitivity. By elevated levels, it is meant that the levels or amount of D2/3 ^{High} receptors in the subject are greater than that in a control, as defined above. Such supersensitivity affects their reaction to dopamine related drugs, for example dopamine agonists, and is a significant consideration in the diagnosis and course of treatment for the subject.

The term “dopamine-related disorder” as used herein refers to any disorder, disease or condition which is the result of modulation of, or causes a modulation in, the activity at a dopamine receptor, in particular the D2/3^{High} receptors. In embodiments of the invention, the dopamine-related disorder is selected from Parkinson's disease, psychoses, schizophrenia, addiction, attention-deficit hyperactivity disorder (ADHD or ADD), adult attention-deficit disorder (AADD), depression, Huntington's disease and Progressive Supranuclear Palsy.

As a representative, non-limiting example, whether or not a Parkinson diseased subject is or is not sensitive to treatment with L-DOPA, or some other dopamine agonist, may depend on the number of high-affinity states of D2 that exist in that particular subject. Likewise, similar determinations are critical in the treatment and diagnosis of psychoses and schizophrenia. In a further aspect of the present invention, the amount or the specific binding of the compound of the invention wherein R¹ is C₁₋₆alkyl in which one hydrogen atom on the alkyl chain is replaced with radioactive fluoro is determined in the globus pallidus and this amount is used to diagnose early stages of Progressive Supranuclear Palsy (PSP). Accordingly, the present invention also includes a method of diagnosing PSP in a subject comprising observing the amount or the specific binding of a compound of the invention wherein R¹ is C₁₋₆alkyl in which one hydrogen atom on the alkyl chain is replaced with radioactive fluoro in the globus pallidus of the subject. In an embodiment of the invention the amount or the specific binding of the compound of the invention wherein R¹ is C₁₋₆alkyl in which one hydrogen atom on the alkyl chain is replaced with radioactive fluoro in the globus pallidus of the subject is compared to a control and if there is an alteration or diminution in the amount or pattern of the specific binding of the compound of the invention wherein R¹ is C₁₋₆alkyl in which one hydrogen atom on the alkyl chain is replaced with radioactive fluoro in the globus pallidus in the subject compared to the control, then the subject may have early stage PSP.

In a further embodiment of the present invention, the amount or the specific binding of D2/3^{High} receptors in a subject's brain is expressed as a percentage of the total population of dopamine D2/3 receptors. This is done by dividing the amount or the specific binding of a compound of the invention wherein R¹ is C₁₋₆alkyl in which one hydrogen atom on the alkyl chain is replaced with radioactive fluoro by the total D2/3 density and multiplying by 100. The total D2/3 density may be determined in humans or animals using any well known method, for example using [¹¹C]raclopride or [³H]raclopride. For example, the total density of D2 receptors in the human caudate nucleus and/or in the putamen is 12 pmol/g (picomoles per gram of original tissue or per mL of pixels) and the total density of D3 receptors in the human caudate nucleus and/or in the putamen is of the order of about 1-2 pmol/g and about 5-6 pmol/g in the human globus pallidus.

The present invention also includes the use of a compound of the invention wherein R¹ is C₁₋₆alkyl in which one hydrogen atom on the alkyl chain is replaced with radioactive fluoro to determine an amount dopamine D2/3^{High} receptors in vivo as well as the use of a compound of the invention wherein R¹ is C₁₋₆alkyl in which one hydrogen atom on the alkyl chain is replaced with radioactive fluoro to prepare a medicament to determine an amount dopamine D2/3^{High} receptors in vivo.

Further, the present invention includes the use of a compound of the invention wherein R¹ is C₁₋₆alkyl in which one hydrogen atom on the alkyl chain is replaced with radioactive fluoro to determine an extent of dopamine supersensitivity in vivo as well as the use of a compound of the invention wherein R¹ is C₁₋₆alkyl in which one hydrogen atom on the alkyl chain is replaced with radioactive fluoro to prepare a medicament to determine an extent of dopamine supersensitivity in vivo.

The present invention also includes a method for conducting positron emission tomography of a subject comprising administering to the subject an effective amount of a compound of Formula I wherein R¹ is C₁₋₆alkyl in which one hydrogen atom on the alkyl chain is replaced with radioactive fluoro and measuring the distribution within the subject of the compound by PET.

The compounds of the invention are suitably formulated into pharmaceutical or radiopharmaceutical compositions for administration to human subjects in a biologically compatible form suitable for administration in vivo. Accordingly, in another aspect, the present invention provides a pharmaceutical or radiopharmaceutical composition comprising a compound of the invention in admixture with a suitable diluent or carrier.

In accordance with the methods of the invention, the compounds of the invention may be administered to a patient in a variety of forms depending on the selected route of administration, as will be understood by those skilled in the art. The compositions of the invention may be administered, for example, by intraveneous administration and the radiopharmaceutical compositions formulated accordingly, for example together with any physiologically and radiologically tolerable vehicle appropriate for administering the compound systemically.

In an embodiment of the invention, the compounds are administered intravenously to minimize metabolism before the compound enters the brain. For imaging purposes, the amount or dosage of compound of the invention wherein R¹ is C₁₋₆alkyl in which one hydrogen atom on the alkyl chain is replaced with radioactive fluoro required to image or quantify the D2^{High} in the brain will be readily ascertained by one of ordinary skill in the nuclear medicine art taking into account the specific activity of the compound and the radiation dosimetry. As is known by those skilled in the nuclear medicine art, the number of milliCuries of the radiolabeled compound to be administered for the PET or SPECT scan will be limited by the dosimetry, whereas the mass of compound to be administered (e.g. μg/kg or mg/kg of body weight of the patient) is calculated based on the specific activity of the synthesized compound, i.e., the amount of radioactivity/mass, of radiolabeled compound. It will be appreciated that because of the short half-life of the radioisotopes, it is often necessary to make the radiolabeled compound at or near the site of administration. The specific activity of the compounds must then be ascertained in order to calculate the proper dosing. Such techniques are well known to those skilled in the art.

By way of illustration, and not in limitation, the amount of a compound of the invention wherein R¹ is C₁₋₆alkyl in which one hydrogen atom on the alkyl chain is replaced with radioactive fluoro to be administered to a human subject is a minimum of 10 milliCuries (mCi), administered intravenously. The amount of a compound of the invention wherein R¹ is C₁₋₆alkyl in which one hydrogen atom on the alkyl chain is replaced with radioactive fluoro to be administered to a rat may be a minimum of 0.5 mCi. The maximum amount of a compound of the invention wherein R¹ is C₁₋₆alkyl in which one hydrogen atom on the alkyl chain is replaced with radioactive fluoro would be that amount that would be harmful or toxic to the subject. In an embodiment of the invention the compound of the invention wherein R¹ is C₁₋₆alkyl in which one hydrogen atom on the alkyl chain is replaced with radioactive fluoro is administered using a bolus infusion protocol (R. E. Carson et al., J. Cereb. Blood Flow Metab. 17: 437-447, 1997), with 60% of the dose injected as a bolus over 1 min and the rest injected by means of intravenous infusion over 75 min.

The present invention further includes a method of screening for compounds that bind to the D2/3^{High} receptors comprising (a) combining a sample comprising the D2/3^{High} receptors with a compound of the invention wherein R¹ is C₁₋₆alkyl in which one hydrogen atom on the alkyl chain is replaced with radioactive fluoro and test compound under conditions sufficient for binding of the compound and the test compound to the D2/3^{High} receptors; and (b) determining an amount of binding of the compound that is inhibited in the presence of the test compound, wherein the greater the amount of binding of the compound that is inhibited in the presence of the test compound, the greater the binding of the test compound to the D2/3^{High} receptors. The invention also includes the use of compound of the invention wherein R¹ is C₁₋₆alkyl in which one hydrogen atom on the alkyl chain is replaced with radioactive fluoro to screen for compounds that bind to the D2/3^{High} receptors.

In an embodiment of the invention, the compound of the invention wherein R¹ is C₁₋₆alkyl in which one hydrogen atom on the alkyl chain is replaced with radioactive fluoro. In a further embodiment of the invention the compound is selected from [¹⁸F]-4-(3-fluoropropyl)-3,4,4a,5,6,10b-hexahydro-2H-naphtho[1,2-b][1,4]oxazin-9-ol; [¹⁸F]-4-(2-fluoropropyl)-3,4,4a,5,6,10b-hexahydro-2H-naphtho[1,2-b][1,4]oxazin-9-ol; [¹⁸F]-(4aR,10bR)-4-(2-fluoroethyl)-3,4,4a,5,6,10b-hexahydro-2H-naphtho[1,2-b][1,4]oxazin-9-ol; and [¹⁸F]-(4aR,10bR)-4-(3-fluoropropyl)-3,4,4a,5,6,10b-hexahydro-2H-naphtho[1,2-b][1,4]oxazin-9-ol.

The sample comprising the D2/3^{High} receptor may be, for example, any preparation containing cells that express one or both of these receptors, suitably one of these receptor types. The cells may be cells that naturally contain the D2/3^{High} receptors or may be cells that have been transformed specifically product the D2/3^{High} receptors. Such cell lines are well known in the art.

The test compound may be any compound that one wishes to test for binding to the D2/3^{High} receptors and it may be a mixture of compounds, for example, from a combinatorial library.

The present invention also includes a kit for the rapid synthesis of the compounds of the invention wherein R¹ is C₁₋₆alkyl in which one hydrogen atom on the alkyl chain is replaced with radioactive fluoro. The kit includes a compound selected from a compound of Formula VII and a compound of Formula XI, and protected derivatives thereof: 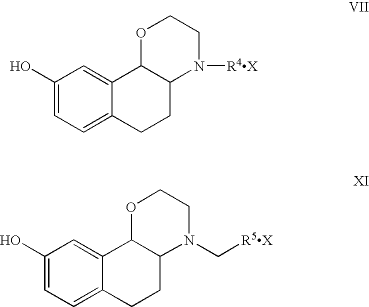
wherein R⁴ is C₁₋₆alkylene, R⁵ is C₁₋₅alkylene and X is a suitable leaving group, such as tosyl, which is capable of being displaced by a radioactive fluoride anion. The protected derivatives include compounds of Formula VII and XI having a suitable protecting group on the hydroxyl group. Suitable protecting groups would be known to those skilled in the art, for example with reference to “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999. For example, suitable protecting groups are stable to basic conditions, for example alkyl ether protecting groups, such as methoxy methyl (MOM).

Optionally, the kit can include items of apparatus, such as a reaction vessel, device for transferring isotopic material to the reaction vessel, pre-packed separation column for separating product from excess reactants, shielding, reaction solvents and the like, as known in the art. See, e.g. Zea-Ponce, U., et al (1998) J. Nuclear Med. 36:525-529.

In another of its aspects, the present invention also includes a method of treating a dopamine-related disorder comprising administering to a subject in need thereof, an effective amount of a compound of Formula I′: 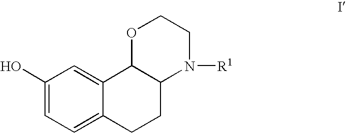
and pharmaceutically acceptable salts and solvates thereof,
wherein R¹ is C₁₋₆alkyl in which one hydrogen atom on the alkyl chain is replaced with fluoro.

Also included is a use of a compound of Formula I′, and pharmaceutically acceptable salts and solvates thereof, to treat a dopamine-related disorder and a use of a compound of Formula I′, and pharmaceutically acceptable salts and solvates thereof, to prepare a medicament to treat a dopamine related disorder.

The compounds of Formula I′ include those in which R¹ is C₁₋₆alkyl in which one hydrogen atom on the alkyl chain is replaced with fluoro. Accordingly, in an embodiment of the invention R¹ is selected from the group consisting of methyl, ethyl, n-propyl, isopropyl, n-butyl, s-butyl, isobutyl, t-butyl, n-pentyl, isopentyl, neopentyl, t-pentyl, 3-methylbutan-2-yl, 2-methylbutan-1-yl, n-hexyl, hexan-2-yl, 2-methylpentan-2-yl, 2,3-dimethylbutan-2-yl, 3,3-dimethylbutan-2-yl, 3-methylpentan-2-yl, 4-methylpentan-2-yl, 2,3-dimethylbutan-1-yl, 3,3-dimethylbutan-1-yl, hexan-2-yl, 2-methylpentan-1-yl, 3-methylpentan-1-yl, 4-methylpentan-1-yl and hexan-3-yl, wherein one hydrogen atom on R¹ is replaced with fluoro or radioactive fluoro. In a further embodiment of the invention, R¹ is selected from the group consisting of methyl, ethyl, n-propyl, isopropyl, n-butyl, s-butyl, isobutyl and t-butyl, wherein one hydrogen atom on R¹ is replaced with fluoro. In a still further embodiment of the invention, R¹ is selected from the group consisting of methyl, ethyl, n-propyl, n-butyl, n-pentyl and n-hexyl, wherein one hydrogen atom on R¹ is replaced with fluoro. In another further embodiment of the invention, R¹ is selected from the group consisting of ethyl, n-propyl and n-butyl, wherein one hydrogen atom on R¹ is replaced with fluoro. In yet another further embodiment of the invention, R¹ is selected from the group consisting of ethyl and n-propyl, wherein one hydrogen atom on R¹ is replaced with fluoro. In another embodiment of the invention R¹ is n-propyl wherein one hydrogen atom on R¹ is replaced with fluoro.

In an embodiment of the invention, the hydrogen on R¹ that is replaced with fluoro in the compound of Formula I′ is a hydrogen atom on a terminal carbon atom. The term “terminal carbon atom” as used herein refers to the carbon atom in R¹ that is furthest away from the nitrogen atom to which R¹ is attached.

In a further embodiment of the invention, the compound of Formula I′ is selected from the group consisting of:
4-(3-fluoropropyl)-3,4,4a,5,6,10b-hexahydro-2H-naphtho[1,2-b][1,4]oxazin-9-ol;
4-(2-fluoroethyl)-3,4,4a,5,6,10b-hexahydro-2H-naphtho[1,2-b][1,4]oxazin-9-ol;
4-(4-fluorobutyl)-3,4,4a,5,6,10b-hexahydro-2H-naphtho[1,2-b][1,4]oxazin-9-ol;
4-(5-fluoropentyl)-3,4,4a,5,6,10b-hexahydro-2H-naphtho[1,2-b][1,4]oxazin-9-ol;
4-(6-fluorohexyl)-3,4,4a,5,6,10b-hexahydro-2H-naphtho[1,2-b][1,4]oxazin-9-ol;
(4aR,10bR)-4-(2-fluoroethyl)-3,4,4a,5,6,10b-hexahydro-2H-naphtho[1,2-b][1,4]oxazin-9-ol;
(4aR,10bR)-4-(3-fluoropropyl)-3,4,4a,5,6,10b-hexahydro-2H-naphtho[1,2-b][1,4]oxazin-9-ol;
(4aR,10bR)-4-(4-fluorobutyl)-3,4,4a,5,6,10b-hexahydro-2H-naphtho[1,2-b][1,4]oxazin-9-ol;
(4aR,10bR)-4-(5-fluoropentyl)-3,4,4a,5,6,10b-hexahydro-2H-naphtho[1,2-b][1,4]oxazin-9-o; and
(4aR,10bR)-4-(6-fluorohexyl)-3,4,4a,5,6,10b-hexahydro-2H-naphtho[1,2-b][1,4]oxazin-9-ol,
and pharmaceutically acceptable salts and solvates thereof.

The compounds of Formula I′ may be prepared using the method described above for the preparation of the compounds of Formula I and Ia.

The term “effective amount of a compound of Formula I′″ is that amount sufficient to effect beneficial or desired results, including clinical results, and, as such, an “effective amount” depends upon the context in which it is being applied. For example, in the context of administering an agent that is treats dopamine-related disorders, an effective amount of an agent is, for example, an amount sufficient to achieve such a treatment as compared to the response obtained without administration of the agent.

The term “dopamine-related disorder” as used herein refers to any disorder, disease or condition which is the result of modulation of, or causes a modulation in, the activity at a dopamine receptor, in particular the D2/3^{High} receptors. In embodiments of the invention, the dopamine-related disorder is selected from Parkinson's disease, psychoses, schizophrenia, addiction, attention-deficit hyperactivity disorder (ADHD or ADD), adult attention-deficit disorder (AADD), depression, Huntington's disease and Progressive Supranuclear Palsy. The compound of Formula I′ may be used to treat one or more dopamine-related disorders. Suitably the dopamine-related disorder is Parkinson's disease.

As used herein, and as well understood in the art, “treating” or “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “prevention” or “prophylaxis”, or synonym thereto, as used herein refers to a reduction in the risk or probability of a patient becoming afflicted with a dopamine-related disorder or manifesting a symptom associated with a dopamine-related disorder.

“Palliating” a disease or disorder, means that the extent and/or undesirable clinical manifestations of a disorder or a disease state are lessened and/or time course of the progression is slowed or lengthened, as compared to not treating the disorder.

The compounds of Formula I′ may be used in the form of the free base, in the form of salts and solvates. All forms are within the scope of the invention.

In accordance with the methods of the invention, the described compounds of Formula I′ or salts and solvates thereof may be administered to a patient in a variety of forms depending on the selected route of administration, as will be understood by those skilled in the art. The compounds Formula I′ may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A compound of Formula I′ may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound of the invention may be incorporated with excipient and used in the form of ingestible tablets, buccal tablets, troches, capsules, elixirs, suspensions, syrups, wafers, and the like.

A compound Formula I′ may also be administered parenterally. Solutions of a compound of the invention can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2000-20th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The compounds of Formula I′ can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of Formula I′ may also be delivered by the use of monoclonal antibodies as individual carriers to which the compound molecules are coupled. The compounds of Forumla Ib may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of Formula I′ may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound of Formula I′ may be used alone, in combination with another compound of Formula I′ and/or pharmaceutical salts and/or solvates thereof, or in combination with other known agents useful for treating or preventing dopamine-related disorders.

When used in combination with other agents useful in treating dopamine-related disorders, the compound of Formula I′ is suitably administered contemporaneously with those agents. As used herein, “contemporaneous administration” of two substances to an individual means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances.

The compounds of Formula I′ may be administered to an animal alone or also in combination with pharmaceutically acceptable carriers, as noted above, the proportion of which is determined by the solubility and chemical nature of the compound, chosen route of administration and standard pharmaceutical practice.

The dosage of the compound of Formula I′, and/or compositions comprising a compound of Formula I′, can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The compound of Formula I′ may be administered initially in a suitable dosage that may be adjusted as required, depending on the clinical response. As a representative example, oral dosages of the compound of Formula I′, will range between about 0.02 μg/kg of body weight per day (mg/kg/day) to about 0.5 μg/kg/day. When formulated for oral administration, the compounds are suitably in the form of tablets containing 0.01, 0.05, 0.1, 0.5, 1.0, 2.5, 5.0 or 10.0 μg of active ingredient per tablet. The compound of Formula I′ may be administered in a single daily dose or the total daily dose may be divided into two, three of four daily doses. If the compounds of Formula I′ are to be administered transdermally, using, for example, those forms of transdermal skin patches that are well known to those skilled in the art, the dosage administration will be continuous rather than intermittent throughout the dosage range.

The following non-limiting examples are illustrative of the present invention:

### EXAMPLES

### Example 1

### Preparation of (4aR,10bR)-4-(3-Fluoropropyl)-3,4,4a,5,6,10b-hexahydro-2H-naphtho[1,2-b][1,4]oxazin-9-ol Hydrochloride ((+)-F-PHNO-Ic).

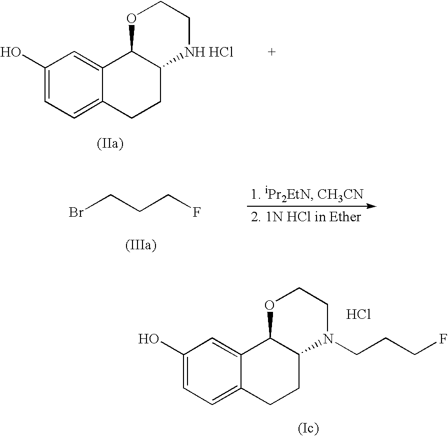

A solution of compound IIa (0.038 g, 0.157 mmol) in dry acetonitrile (1.5 mL) was treated with diisopropyl ethylamine (0.1 mL) followed by 3-bromo-fluoropropane (0.1 mL) at room temperature and the resulting mixture was stirred at 70° C. for 48 hours. The reaction was brought to room temperature and the solvent was evaporated. The crude product was dissolved into ethyl acetate (20 mL), washed with saturated NaHCO₃ solution (10 mL),brine (10 mL) and dried (Na₂SO₄). The ethyl acetate was evaporated and the crude product was purified by column chromatography (2M NH₃ in MeOH:CH₂Cl₂ 5:95) to obtain the free base.

A solution of the above free base in methanol (1 nL) was treated with 1N HCl in ether (0.5 mL) and was stirred for 15 minutes at room temperature. The solvent was evaporated and the crude product was crystallized from ethanol/ether to obtain compound Ic (0.47 g, quantitative). ¹H NMR (DMSO-d₆) δ 1.80-1.86 (m, 1H), 2.05-2.27 (m, 2H), 2.40-2.46 (m, 2H), 2.79-2.82 (m, 2H), 3.07-3.27 (m, 2H), 3.48-3.61 (m, 2H), 4.10-4.25 (m, 2H), 4.51 (t, 1H, J=5.4 Hz), 4.66 (t, 1H, J=5.4 Hz), 4.83 (d, 1H, J=9.3 Hz), 6.65 (dd, 1H, J=2.4, 8.4 Hz), 6.84 (d, 1H, J=2.4 Hz), 6.94 (d, 1H, J=8.4 Hz), 9.30 (s, 1H), 11.38 (s, 1H); ESI-MS (m/z, %) 266 (MH⁺, 100, free base); ESI-HRMS calculated for C₁₅H₂₁NO₂F (MH+, free base): 266.1559; observed: 266.1550; m.p. 253-255° C.

### Example 2

### Preparation of (4aR,10bR)-4-(2-Fluoroethyl)-3,4,4a,5,6,10b-hexahydro-2H-naphtho[1,2-b][1,4]oxazin-9-ol Hydrochloride ((+)-F-EHNO-Id).

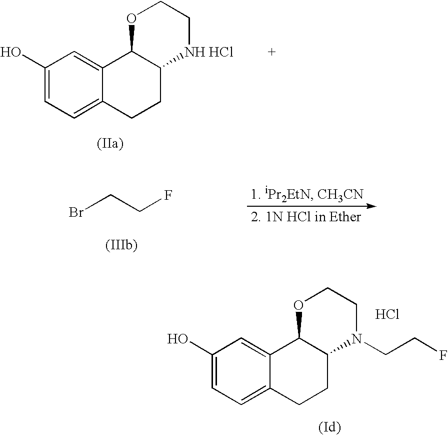

A stirred suspension of compound IIa (70 mg. 0.41 mmol) and diisopropylethyl amine (100 μl) in acetonitrile (2 mL) was treated with 2-fluoroethylbromide (IIb, 100 μL). The mixture was heated to 70° C. for 2 days. The residue after evaporation of the acetonitrile was taken up in ethyl acetate (20 mL), washed with aqueous sodium bicarbonate, dried (Na₂SO₄), and filtered. The oily solid obtained from evaporation of solvent was re-dissolved in ethyl acetate and passed through a short plug of silica gel. Treatment with ethereal HCl (1N) afforded the hydrochloride salt (Ib) as a white solid which was recrystallized from ethanol as white needles (58 mg) Anal. C, 58.43; H, 6.66; Cl, 12.32; F, 6.60; N, 4.87; 0, 11.12Anal. Calcd for C₁₄H₁₉ClFNO₂ C, 58.43; H, 6.66: N, 4.87. Found: C, 58.40; H, 6.68; N, 4.78.

In a like manner the following additional compounds were prepared:
(4aR,10bR)-4-(4-Fluorobutyl)-3,4,4a,5,6,10b-hexahydro-2H-naphtho[1,2-b][1,4]oxazin-9-ol Hydrochloride ((+)-F-But-HNO-Ie);
(4aR,10bR)-4-(5-Fluoropentyl)-3,4,4a,5,6,10b-hexahydro-2H-naphtho[1,2-b][1,4]oxazin-9-ol Hydrochloride ((+)-F-Pent-HNO-If); and
(4aR,10bR)-4-(6-Fluorohexyl)-3,4,4a,5,6,10b-hexahydro-2H-naphtho[1,2-b][1,4]oxazin-9-ol Hydrochloride ((+)-F-Hex-HNO-Ig).

### Example 3

### Preparation of (+)-[¹⁸F]-PHNO

[¹⁸F]-Propionyl chloride/THF is trapped in a 5 ml V-vial containing (+)-3,4,4a,5,6,10b-hexahydro-2H-naphtho[1,2-b][1,4]oxazin-9-olhydrochloride (8 μmoles), DIPEA (50 μl), and THF (50 μl) at <-30° C. 60° C. (about 2.5 min). One min later the vial is then cooled in an ethanol/dry ice bath until the internal temperature was <-30° C. at which point LiAlH₄ in THF was added (0.2N, 0.6 mL). The vial is then re-immersed in the oil bath and THF removed by a flow of N₂ (80 mL/min) through the vial. Upon evaporation of all the THF, aqueous HCl (0.6N, 0.8 mL) is added followed after 30 sec by 1 ml of HPLC eluent. The reaction mixture is purified by reverse-phase HPLC. The desired fraction is collected, evaporated to dryness under vacuum at 70° C., and the residue taken up in 10 mL of sterile saline. The saline solution of [¹⁸F]-(+)PHNO is passed through a sterile 0.22 μm filter into a sterile, pyrogen-free bottle containing aqueous sodium bicarbonate (1 mL, 8.4%). Aliquots of the formulated solution are used to establish the chemical and radiochemical purity and specific activity of the final solution by analytical HPLC. The identity of the product is confirmed by HPLC and radio-thin layer chromatography (radio-TLC). Radio-TLC of the formulated product is carried out on silica plates.

### Example 4

### Preparation of [¹⁸F]-4-(2-fluoroethyl)-3,4,4a,5,6,10b-hexahydro-2H-naphtho[1,2-b][1,4]oxazin-9-ol

[¹⁸F]-2-Fluoroethyl bromide (Shiue, C.-Y. et al. J. Labeled Comp. Radiopharm. 24:56, 1987; Wilson, A. A. et al. Appl Radiat Isotopes, 46:765, 1995; Wilson, A. A. et al. Nucl Med Biol, 23:487, 1996) was distilled into a cooled (−40° C.) 5 mL septum-sealed V-vial. This vessel contained a freshly prepared solution of 3,4,4a,5,6,10b-hexahydro-2H-naphtho[1,2-b][1,4]oxazin-9-ol (5 mg), DIPEA (50 μL) and DMF (0.2 mL). Upon trapping the maximum radioactivity (≈4 min), the needles were removed, the vial heated to 80° C. for 8 min, and then volatiles removed by a nitrogen sweep (200 mL/min for 90 sec). The residue was cooled, diluted with 1 mL of HPLC buffer and purified by semi-preparative HPLC. The desired fraction was collected, evaporated to dryness, and the residue dissolved in 10 mL of sterile saline. This solution was passed through a sterile 0.22 μm filter into a sterile, pyrogen free bottle containing aqueous sodium bicarbonate (1 mL, 8.4%). The radiochemical purity and specific activity of the final solution was determined by analytical HPLC and radio-TLC.

### Example 5

### Biological Potency of Compounds of the Invention

The biological potency of selected compounds of the invention was tested on the high-affinity state of dopamine D2 receptors in rat homogenized striatal tissue, using the method of Seeman et al. (Proc. Nat. Acad. Sci. U.S.A. 102: 3513-3518, 2005; and Synapse 49; 209-215, 2003) and on the rat cloned D3 receptor, using 1.6 nM [³H]-raclopride. The results are presented in Table 1.

### Example 5

### Labeling of D2^{High} Receptors In Vivo

For humans, a minimum amount of 10 milliCuries (0.5 mCi for rats) of (500-1,000 mCi/μmole) is injected intravenously in a human volunteer, using a bolus plus infusion protocol (R. E. Carson et al., J. Cereb. Blood Flow Metab. 17: 437-447, 1997), with 60% of the dose injected as a bolus over 1 min and the rest injected by means of intravenous infusion over 75 min.

After a brief transmission scan for attenuation correction of the emission scans, emission scans are obtained every minute for the first 15 min, and then every 5 min until the end of the study at 75 min. The PET scanning is conducted by using a dedicated brain scanner, GEMS PC2048-15B PET camera (General Electric Medical Systems) that produces fifteen 6.5 mm-thick slices with a resolution of 5-6 mm. The volunteer is scanned lying down and the head fixed by using a thermoplastic face mask. For accurate anatomical position of the various brain regions, each volunteer receives an MRI (magnetic resonance imaging) scan. The regions of interest are transferred to the PET images by using Alice™ 3.1 software.

The regions of interest include the head of the caudate nucleus. The peak emission in the caudate nucleus occurs at 10 minutes after the intravenous injection at about 0.22% of the injected dose per kg. Several hours after the initial injection of (+)-[¹⁸F]-PHNO, a second injection of (+)-[¹⁸F]-PHNO is given intravenously at the same time as a very low dose of non-radioactive (+)PHNO, (+)-F-PHNO or a very low dose of apomorphine. The co-administered dose of (+)PHNO, (+)-F-PHNO or apomorphine should be on the order of about 10 to about 50 times the dose of total drug in the (+)-[¹⁸F]-PHNO dose (radiolabeled and non-radiolabeled molecules), thus defining a baseline to determine the number of high affinity states of D2 in the same brain region. The difference between the brain image done without co-injection and the image with cop-injection is defined as specific binding, and indicates the presence of D2^{high} receptors. The density of the D2^{High} sites in the caudate nucleus that are labeled by (+)-[¹⁸F]-PHNO is calculated, knowing the specific activity of the tracer injected and the amount of radioactivity detected by the positron imaging camera.

To confirm that the pattern of (+)-[¹⁸F]-PHNO specific binding to brain tissue truly reflects binding to dopamine D2 receptors, the following experiments are done: A. The effect of reserpine and/or alpha-methyl-paratyrosine is to increase the specific binding, because endogenous dopamine is removed. B. The amount of specific binding is to be found highest in the striatum (caudate nucleus and putamen), with low amount of nonspecific binding in all the other brains regions, including the cerebellum. C. Antipsychotic drugs given prior to the injection of (+)-[¹⁸F]-PHNO reduce the magnitude of specific binding. D. Non-dopaminergic drugs do not affect the specific binding of (+)-[¹⁸F]-PHNO.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1

| Compound | K_{D} (nM, D2^{High}) | K_{D} (nM, D3) |
|---|---|---|
| (+)-F-EHNO | 1.7 ± 0.3 | 1.7 |
| (+)-F-PHNO | 0.46 ± 0.06 | 0.38 |
| (+)-F-But-HNO | 20 ± 8.7 | 34 |
| (+)-F-Pent-HNO | 25 ± 4 | 54 |
| (+)-F-Hex-HNO | 52 ± 27 | 45 |

What is claimed is:

1. A compound selected from a compound of Formula I: 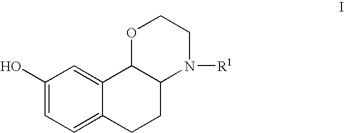
and pharmaceutically acceptable salts and solvates thereof,
wherein R¹ is C₁₋₆alkyl in which one hydrogen atom on the alkyl chain is replaced with fluoro or radioactive fluoro.

2. The compound according to claim 1, wherein R¹ is selected from the group consisting of methyl, ethyl, n-propyl, isopropyl, n-butyl, s-butyl, isobutyl, t-butyl, n-pentyl, isopentyl, neopentyl, t-pentyl, 3-methylbutan-2-yl, 2-methylbutan-1-yl, n-hexyl, hexan-2-yl, 2-methylpentan-2-yl, 2,3-dimethylbutan-2-yl, 3,3-dimethylbutan-2-yl, 3-methylpentan-2-yl, 4-methylpentan-2-yl, 2,3-dimethylbutan-1-yl, 3,3-dimethylbutan-1-yl, hexan-2-yl, 2-methylpentan-1-yl, 3-methylpentan-1-yl, 4-methylpentan-1-yl and hexan-3-yl, and one hydrogen atom on R¹ is replaced with fluoro or radioactive fluoro.

3. The compound according to claim 2, wherein R¹ is selected from the group consisting of methyl, ethyl, n-propyl, isopropyl, n-butyl, s-butyl, isobutyl and t-butyl, and one hydrogen atom on R¹ is replaced with fluoro or radioactive fluoro.

4. The compound according to claim 2, wherein R¹ is selected from the group consisting of methyl, ethyl, n-propyl, n-butyl, n-pentyl and n-hexyl, and one hydrogen atom on R¹ is replaced with fluoro or radioactive fluoro.

5. The compound according to claim 4, wherein R¹ is selected from the group consisting of ethyl, n-propyl and n-butyl, and one hydrogen atom on R¹ is replaced with fluoro or radioactive fluoro.

6. The compound according to claim 5, wherein R¹ is selected from the group consisting of ethyl and n-propyl, and one hydrogen atom on R¹ is replaced with fluoro or radioactive fluoro.

7. The compound according to claim 1, wherein the hydrogen atom on R¹ that is replaced with fluoro or radioactive fluoro is a hydrogen atom on a terminal carbon atom.

8. The compound according to claim 1, wherein the hydrogen atom on R¹ is replaced with radioactive fluoro.

9. The compound according to claim 8, wherein the radioactive fluoro is [¹⁸F].

10. The compound according to claim 1, wherein the hydrogen atom on R¹ is replaced with fluoro.

11. A compound according to claim 1, selected from a compound of Formula Ia: 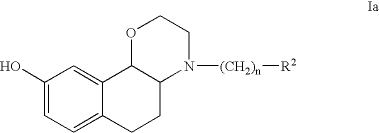
and pharmaceutically acceptable salts and solvates thereof,
wherein n is 1, 2, 3, 4, 5 or 6 and R² is fluoro or radioactive fluoro.

12. The compound according to claim 1 1, wherein n is 1, 2, 3 or 4.

13. The compound according to claim 12, wherein n is 2 or 3.

14. The compound according to claim 13, wherein n is 3.

15. The compound according to claim 11, wherein R² is radioactive fluoro.

16. The compound according to claim 15, where the radioactive fluoro is [¹⁸F].

17. The compound according to claim 11, wherein R² is fluoro.

18. The compound according to claim 1 in the (+)-trans configuration.

19. The compound according to claim 1, selected from the group consisting of:
[¹⁸F]-4-(3-fluoropropyl)-3,4,4a,5,6,10b-hexahydro-2H-naphtho[1,2-b][1,4]oxazin-9-ol;
[¹⁸F]-4-(2-fluoroethyl)-3,4,4a,5,6,10b-hexahydro-2H-naphtho[1,2-b][1,4]oxazin-9-ol;
[¹⁸F]-4-(4-fluorobutyl)-3,4,4a,5,6,10b-hexahydro-2H-naphtho[1,2-b][1,4]oxazin-9-ol;
[¹⁸F]-4-(5-fluoropentyl)-3,4,4a,5,6,10b-hexahydro-2H-naphtho[1,2-b][1,4]oxazin-9-o;
[¹⁸F]-4-(6-fluorohexyl)-3,4,4a,5,6,10b-hexahydro-2H-naphtho[1,2-b][1,4]oxazin-9-ol;
[¹⁸F]-(4aR,10bR)-4-(2-fluoroethyl)-3,4,4a,5,6,10b-hexahydro-2H-naphtho[1,2-b][1,4]oxazin-9-ol;
[¹⁸F]-(4aR,10bR)-4-(3-fluoropropyl)-3,4,4a,5,6,10b-hexahydro-2H-naphtho[1,2-b][1,4]oxazin-9-ol;
[¹⁸F]-(4aR,10bR)-4-(4-fluorobutyl)-3,4,4a,5,6,10b-hexahydro-2H-naphtho[1,2-b][1,4]oxazin-9-ol;
[¹⁸F]-(4aR,10bR)-4-(5-fluoropentyl)-3,4,4a,5,6,10b-hexahydro-2H-naphtho[1,2-b][1,4]oxazin-9-o;
[¹⁸F]-(4aR,10bR)-4-(6-fluorohexyl)-3,4,4a,5,6,10b-hexahydro-2H-naphtho[1,2-b][1,4]oxazin-9-ol;
4-(3-fluoropropyl)-3,4,4a,5,6,10b-hexahydro-2H-naphtho[1,2-b][1,4]oxazin-9-ol;
4-(2-fluoroethyl)-3,4,4a,5,6,10b-hexahydro-2H-naphtho[1,2-b][1,4]oxazin-9-ol;
4-(4-fluorobutyl)-3,4,4a,5,6,10b-hexahydro-2H-naphtho[1,2-b][1,4]oxazin-9-ol;
4-(5-fluoropentyl)-3,4,4a,5,6,10b-hexahydro-2H-naphtho[1,2-b][1,4]oxazin-9-ol;
4-(6-fluorohexyl)-3,4,4a,5,6,10b-hexahydro-2H-naphtho[1,2-b][1,4]oxazin-9-ol;
(4aR,10bR)-4-(2-fluoroethyl)-3,4,4a,5,6,10b-hexahydro-2H-naphtho[1,2-b][1,4]oxazin-9-ol;
(4aR,10bR)-4-(3-fluoropropyl)-3,4,4a,5,6,10b-hexahydro-2H-naphtho[1,2-b][1,4]oxazin-9-ol;
(4aR,10bR)-4-(4-fluorobutyl)-3,4,4a,5,6,10b-hexahydro-2H-naphtho[1,2-b][1,4]oxazin-9-ol;
(4aR,10bR)-4-(5-fluoropentyl)-3,4,4a,5,6,10b-hexahydro-2H-naphtho[1,2-b][1,4]oxazin-9-o; and
(4aR,10bR)-4-(6-fluorohexyl)-3,4,4a,5,6,10b-hexahydro-2H-naphtho[1,2-b][1,4]oxazin-9-ol.

20. The compound according to claim 19, selected from the group consisting of:
[¹⁸F]-4-(3-fluoropropyl)-3,4,4a,5,6,10b-hexahydro-2H-naphtho[1,2-b][1,4]oxazin-9-ol;
[¹⁸F]-4-(2-fluoroethyl)-3,4,4a,5,6,10b-hexahydro-2H-naphtho[1,2-b][1,4]oxazin-9-ol;
[¹⁸F]-(4aR,10bR)-4-(2-fluoroethyl)-3,4,4a,5,6,10b-hexahydro-2H-naphtho [1,2-b][1,4]oxazin-9-ol; and
[¹⁸F]-(4aR,10bR)-4-(3-fluoropropyl)-3,4,4a,5,6, 10b-hexahydro-2H-naphtho[1,2-b][1,4]oxazin-9-ol.

21. The compound according to claim 19, selected from the group consisting of:
4-(3-fluoropropyl)-3,4,4a,5,6,10b-hexahydro-2H-naphtho[1,2-b][1,4]oxazin-9-ol;
4-(2-fluoroethyl)-3,4,4a,5,6,10b-hexahydro-2H-naphtho[1,2-b][1,4]oxazin-9-ol;
(4aR,10bR)-4-(2-fluoroethyl)-3,4,4a,5,6,10b-hexahydro-2H-naphtho[1,2-b][1,4]oxazin-9-ol; and
(4aR,10bR)-4-(3-fluoropropyl)-3,4,4a,5,6,10b-hexahydro-2H-naphtho[1,2-b][1,4]oxazin-9-ol.

22. A method of labeling dopamine D2/3 receptors in vivo comprising administering an effective amount of a compound according to claim 8, 9, 15, 16 or 20, to a subject in need thereof, and imaging an amount the compound in said subject.

23. The method according to claim 22, wherein the amount of the compound is imaged in the brain of said subject.

24. A method for conducting positron emission tomography (PET) of a subject comprising administering to the subject an effective amount of a compound according to claim 8 and measuring distribution within the subject of the compound by PET.

25. The method according to claim 22, wherein the subject is a human.

26. A method for determining an amount of dopamine D2/3^{High} receptors comprising administering an effective amount of a compound according to claim 8 to a subject and determining an amount or a specific binding of the compound in the subject's brain, wherein the amount or the specific binding of the compound in the subject's brain is indicative of the amount of dopamine D2/3^{High} receptors.

27. The method according to claim 25, wherein the amount or the specific binding of the compound the subject's brain is correlated with the amount of dopamine D2/3^{High} receptors in the brain, such that the greater the amount or the specific binding of the compound, the greater the number of D2/3^{High} receptors.

28. The method according to claim 26, wherein the amount or the specific binding of the compound in the brain of the subject is compared to a control and if the amount or the specific binding is greater in the subject compared to the control then the subject is in a state of dopamine supersensitivity.

29. The method according to claim 28, wherein the control is an amount or a specific binding of the compound in a brain under normal conditions.

30. The method according to claim 29, wherein normal conditions include absence of factors that affect the amount of D2/3^{High} receptors in the brain.

31. The method according to claim 30, wherein the factors that affect the amount of D2/3^{High} receptors in the brain are selected from one or more of disease, injury, medication and substance abuse.

32. The method according to claim 28, wherein the control is a subject that does not have any psychiatric illness of drug-induced illness.

33. The method according to claim 26, wherein the subject is a human.

34. The method according to claim 26, wherein the amount or the specific binding of the compound is determined in one or more regions of the brain wherein the regions are selected from the striatum, the nucleus caudate, the putamen and the globus pallidus.

35. The method according to claim 26, wherein the amount or the specific binding of the compound is determined or observed using positron emission tomography (PET).

36. A method of determining an extent of dopamine supersensitivity in a subject comprising administering an effective amount of a compound according to claim 8 to a subject and determining an amount or a specific binding of the compound in the subject's brain, wherein the greater the amount or the specific binding of the compound the greater the number of D2/3^{High} receptors, and wherein the greater the number of D2/3^{High} receptors, the greater the extent of dopamine supersensitivity.

37. The method according to claim 26, wherein the specific binding the compound is determined by:
(a) administering an effective amount of the compound to a subject and observing an amount or density of the compound in the subject's brain;
(b) allowing a suitable amount of time to pass for spontaneous decay of the compound administered in (a);
(c) contemporaneously administering an effective amount of the compound and an effective amount of a suitable non-radiolabelled dopamine agonist or dopamine-mimetic and observing an amount or density the compound in the subject's brain; and
(d) determining a difference between the amount or density of the compound in (a) and the amount or density of the compound in (c), wherein said difference is the specific binding of the compound.

38. The method according to claim 37, wherein the amount or density of radiolabelled compound in (a) and (c) is determined in the same regions of the subject's brain.

39. The method according to claim 38, wherein the amount or density of the radiolabelled compound is observed in the striatum, the caudate nucleus, the putamen regions and/or the globus pallidus.

40. The method according to claims 27, wherein the dopamine agonist or mimetic is selected from a compounds according to claim 10, (+)-PHNO, amphetamine, apomorphine, N-propyl-norapomorphine and dihydroxy-2-dimethyl-aminotetralin.

41. The method according to claim 40 wherein the dopamine agonist or mimetic is is F-(+)PHNO, (+)PHNO, apomorphine or N-propyl-norapomorphine.

42. The method according to claim 41, wherein the dopamine agonist or mimetic is F-(+)PHNO, (+)PHNO or apomorphine.

43. The method according to claim 37, wherein the effective amount of the contemporaneously administered dose of dopamine agonist or mimetic is about 10 to about 50 times the effective amount the compound, including radiolabeled and non-radiolabeled molecules.

44. The method according to claim 26, wherein the amount or specific binding is expressed as a percentage of total population of dopamine D2 receptors.

45. The method according to claim 36, wherein the extent of dopamine supersensitivity is used to assess, treat and/or follow the progress of a dopamine-related disorder.

46. The method according to claim 45, wherein the dopamine related disorder is selected from Parkinson's disease, psychoses, schizophrenia, addiction, attention-deficit hyperactivity disorder (ADHD or ADD), adult attention-deficit disorder (AADD), depression, Huntington's disease and Progressive Supranuclear Palsy.

47. A pharmaceutical or radiopharmaceutical composition comprising a compound according to claim 1, in admixture with a pharmaceutically acceptable diluent or carrier.

48. A method of treating a dopamine-related disorder comprising administering to a subject in need thereof, an effective amount of a compound according to claim 10.

49. The method according to claim 48, wherein the dopamine-related disorder is selected from Parkinson's disease, psychoses, schizophrenia, addiction, attention-deficit hyperactivity disorder (ADHD or ADD), adult attention-deficit disorder (AADD), depression, Huntington's disease and Progressive Supranuclear Palsy.

50. The method according to claim 49, wherein the dopamine-related disorder is Parkinson's disease.

51. A kit for the rapid synthesis of a compound according to claim 8 comprising a compound selected from a compound of Formula VII and a compound of Formula XI, and protected derivatives thereof: 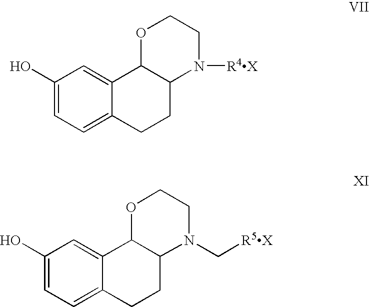
wherein R⁴ is C₁₋₆alkylene, R⁵ is C₁₋₅alkylene and X is a leaving group which is displaced by a radioactive fluoride anion.

52. The kit according to claim 51, further comprising one or more of a reaction vessel, device for transferring isotopic material to the reaction vessel, pre-packed separation column for separating product from excess reactants, shielding and reaction solvents.

53. A method of screening for compounds that bind to the D2/3^{High} receptors comprising (a) combining a sample comprising the D2/3^{High} receptors with a compound according to claim 8 and test compound under conditions sufficient for binding of the compound and the test compound to the D2/3^{High} receptors; and (b) determining an amount of binding of the compound that is inhibited in the presence of the test compound, wherein the greater the amount of binding of the compound that is inhibited in the presence of the test compound, the greater the binding of the test compound to the D2/3^{High} receptors.
